(19) United States
(12) Patent Application Publication
(10) Pub. No.: US 2011/0171193 A1
(43) Pub. Date: Jul. 14, 2011

(54) COMPOSITIONS AND METHODS FOR TREATING PULMONARY HYPERTENSION

(75) Inventors: You-Yang Zhao, Grayslake, IL (US); Asrar B. Malik, Hinsdale, IL (US)

(73) Assignee: THE BOARD OF TRUSTEES OF THE UNIVERSITY OF ILLINOIS, Urbana, IL (US)

(21) Appl. No.: 12/996,712

(22) PCT Filed: Jun. 12, 2009
(86) PCT No.: PCT/US09/47241

§ 371 Date: Dec. 7, 2010

Related U.S. Application Data
(60) Provisional application No. 61/060,831, filed on Jun. 12, 2008.
(60) Provisional application No. 61/182,457, filed on May 29, 2009.

(51) Int. Cl. A61K 31/555; A61K 31/522; A61K 31/352; A61K 31/34; A61K 31/195; A61K 31/33; A61K 31/05; A61K 31/19; A61K 31/445; A61K 38/44; A61K 31/075; A61K 31/56; A61K 31/12; A61K 31/03; A61K 33/26; A61K 33/34; A61K 31/405; A61K 31/192; A61K 31/20; A61K 33/40; A61K 38/00; A61K 31/708; A61K 31/497; A61K 31/505; A61K 31/4985; G01N 33/53; A61P 9/12

(52) U.S. Cl. 424/94.4; 514/263.33; 514/456; 514/474; 514/562; 514/185; 514/183; 514/731; 514/570; 514/315; 514/714; 514/182; 514/678; 514/754; 424/646; 424/630; 514/415; 514/568; 514/560; 424/616; 514/565; 514/15.7; 514/48; 514/569; 514/252.16; 514/275; 514/250; 436/501

(57) ABSTRACT

Provided herein are methods and materials for treating pulmonary hypertension (PH) in a subject. Also provided herein is a method of diagnosing whether a has PH by detecting a PH marker. A PKG pulmonary hypertension marker has been identified and may be useful in predicting PH disease progression and assessing a subject's response to PH therapy.

### CROSS-RELATED APPLICATIONS

The present application claims the benefit of the filing dates of provisional applications 61/182,457, filed on May 29, 2009, and 61/060,831, filed Jun. 12, 2008, which are both incorporated by reference in their entirety.

### FEDERALLY SPONSORED RESEARCH

This invention was made with government support under National Heart, Lung and Blood Institute grant number PO1 HL060678 and National Heart, Lung and Blood Institute grant number R01 HL085462. The government has certain rights in the invention.

### FIELD OF THE INVENTION

The present invention relates to the use of therapeutic agents that target the molecular mechanisms of pulmonary hypertension.

### BACKGROUND

Pulmonary hypertension (PH) is generally characterized by progressive increases in pulmonary vascular resistance (PVR) leading to right ventricular failure, and ultimately to death within 2-3 years after diagnosis. PH has different etiologies that share several pathological defects of the pulmonary microvasculature: worsening vasoconstriction, remodeling of pulmonary vessels, and thrombosis. These changes increase medial thickness, occlude small pulmonary arteries, and result in formation of plexiform lesions, which contribute to increased PVR.

Early assessment of PH may present the best opportunity for treatment intervention. With a better understanding of the molecular mechanisms underlying PH, it becomes possible to identify agents that may be used to treat and/or diagnose PH. There remains a need to identify one or more molecular components that have a causal connection to PH, and therapeutic agents that inhibit, decrease, enhance, or activate those molecular components in PH patients. The molecular components may represent known or unknown proteins or nucleic acids, which may be useful in diagnosing PH, and whose activities or products may be targeted for early intervention therapy.

### SUMMARY OF THE INVENTION

Provided herein is a method for treating a subject having pulmonary hypertension (PH). PH may be pulmonary arterial hypertension or idiopathic pulmonary arterial hypertension. The pulmonary hypertension may be secondary to another disease, such as pulmonary fibrosis or scleroderma. An agent, such as a PKG-effector agent, may be administered to the subject. The PKG-effector agent may be a peroxynitrate scavenger, superoxide scavenger, flavonoid, NOS inhibitor, protein kinase G activator, protein kinase G enhancer, a NADPH oxidase inhibitor, a superoxide dismutase activator, a peroxidase activator, a catalase activator, an antioxidant, and/or combinations thereof. The peroxynitrate scavenger may be uric acid, a plant extracted proanthocyanidin, ascorbate, trolox, glutathione (GSH), Mn (III) tetrakis (4-benzoic acid) porphyrin (MnTBAP), flavonoid, ebselen, catchol (1,2-dihydroxybenzene), kaempferol, galangin, caffeic acid, o-coumaric acid, p-coumaric acid, gallic acid, and ferulic acid. The proanthocyanidin may be extracted from an arborescent and/or herbaceous plant species. The superoxide scavenger may be manganese (III) tetrakis (1-methyl-4-pyridyl) porphyrin pentachloride (MnTMPyP), 1-oxyl-2,2,6,6-tetramethyl-4-hydroxypiperidine (TEMPOL), NAD(P)H:quinone oxidoreductase 1, or any combination thereof. The flavonoid may be quercetin, rutin, morin, acacetin, hispidulin, hesperidin, naringin, or any combination thereof. The NOS inhibitor may be N omega-nitro-L-arginine, N omega-monomethyl-L-arginine, 1-N^{G} monomethyl arginine (1-NMMA), a caveolin-1 peptide, ARL 17477, KLYP956, or any combination thereof. The caveolin-1 peptide may have the sequence DGIWKASFTTFTVTKYWFYR (SEQ ID NO:1). The PKG activator or enhancer may be phosphatidylinositol-3,4,5-trisphosphate (PtdIns(3,4,5)P3), cyclic guanosine 3′, 5′-monophosphate (cGMP), 8-pCPT-cGMP (cGMP derivative), cGMP phosphodiesterase inhibitor, or any combination thereof. The cGMP phosphodiesterase inhibitor may be sulindac sulfone, sildenafil, tadalafil, and/or OSI-461. The superoxide dismutase activator may be a lipid peroxide, a reduced glutathione, and/or a 17β-estradiol. The NADPH oxidase inhibitor may be apocynin and/or diphenylene iodonium. The peroxidase activator may be iron, copper, melatonin, N-acetylcysteine (NAC), and/or 4-hydrobenzoic acid. The catalase activator may be an oxidized linoliec acid such as one or more of 13-hydroperoxy-9,11-octadecadienoic acid (13-HPODE), 13-hydroxy-9,11-octadecadienoic acid (13-HODE), hydrogen peroxide, and oxidized LDL.

One may administer a phosphodiesterase type-5 inhibitor (PDE5 inhibitor) before, after, or at the same time as administering the PKG-effector agent to the subject in need thereof. The PDE5 inhibitor may be sildenafil, avanafil, tadalafil, acetildenafil, CGMP specific phosphodiesterase type-5, udenafil, vardenafil, or any combination thereof.

One may administer an endothelin receptor antagonist before, after, or at the same time as administering the PKG-effector agent to the subject in need thereof. The endothelin receptor antagonist may be atrasentan, bosentan, sitaxsentan, ambrisenten, or any combination thereof.

Any agent, activator, inhibitor, or compound described herein may be administered systemically, orally, by inhalation, parenteral, nasally, vaginally, rectally, sublingually, topically, or any combination thereof. The agent, activator, inhibitor, or compound may be formulated as a capsule, tablet, an elixir, a suspension, a dry powder, an aerosol, a syrup, or any combination thereof.

Also provided herein is a method of diagnosing pulmonary hypertension in a subject. A subject having PH may have a PH marker, such as nitrated PKG. One or more antibodies may be provided that bind to nitrated PKG. The one or more antibodies may then be contacted with a sample from the subject, wherein the subject as having pulmonary hypertension if the one or more antibodies bind to nitrated PDG and is/are detected in the sample. The nitrated PKG may be detected using an antibody capable of binding the one or more antibodies that are bound to nitrated PKG.

Also provided herein is a kit, which may be used for diagnosing, monitoring, or treating PH. The kit may have a sample collecting means, a means for determining the presence or absence of a PH-marker, a PH-marker for use as a positive control, and/or a PH-marker detection means. The detection means may include substrates, such as filter paper, and protein purification reagents. The marker detection means may include primary and secondary antibodies and one or more buffers. The kit may also comprise a control sample. The control sample may not comprise a PH-marker.

### BRIEF DESCRIPTION OF THE DRAWINGS

FIG. 1 shows increased pulmonary arterial pressure and vascular resistance in Cav1^{-/-} mice can be rescued in DKO mice.

FIG. 2 shows the prevention of pulmonary tissue remodeling induced by Cav1 deletion in DKO mice.

FIG. 3 shows impaired PKG activity secondary to tyrosine nitration in Cav1^{-/-} lungs.

FIG. 4 shows the result of PKG1 tyrosine nitration and impaired kinase activity in human pulmonary artery smooth muscle cells following SIN-1 treatment.

FIG. 5 shows inhibition of eNOS with L-NAME ad libitum reverses PH in Cav1^{-/-} mice.

FIG. 6 shows quantitative analyses of IGF-I and VEGF-A expression in mouse lungs.

FIG. 7 shows L-NAME treatment of Cav1^{-/-} mice rescues lung pathology.

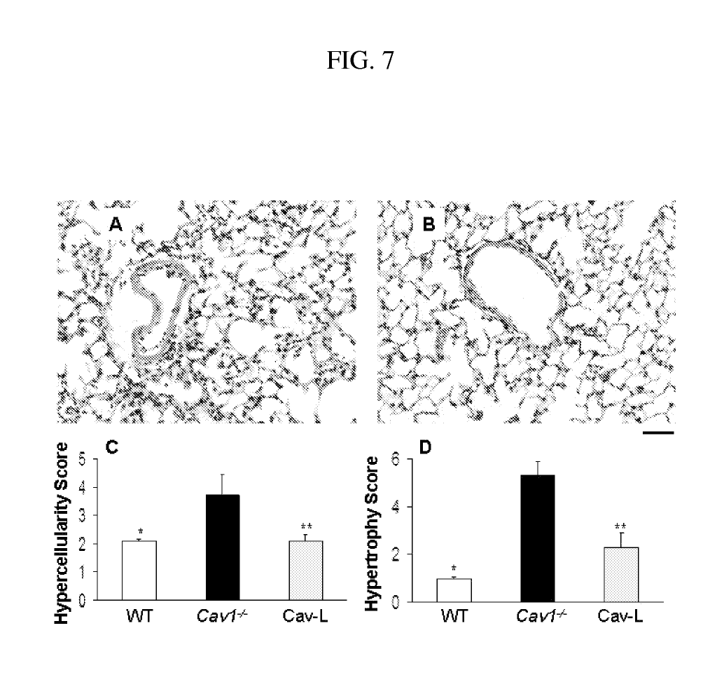

FIG. 8 shows the identification of PKG-1α tyrosine 124 as a target for nitration responsible for impairment of PKG kinase activity.

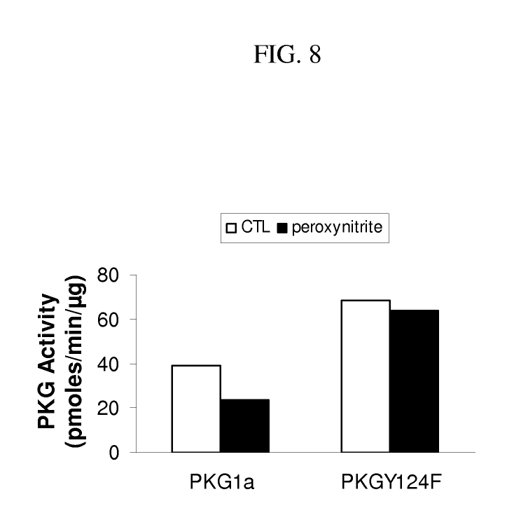

FIG. 9 shows the identification of target tyrosine residues responsible for the impairment of PKG kinase activity upon nitration.

FIG. 10 shows tyrosine-induced impairment of PKG activity mediates pulmonary hypertension in Cav1^{-/-} mice.

FIG. 11 shows PKG tyrosine nitration in lung tissue from IPAH subjects.

FIG. 12 shows similar iNOS and nNOS expression and iNOS-derived NO production in WT and Cav1^{-/-} mouse lungs.

FIG. 13 shows normal sGC activity and cGMP production in Cav1^{-/-} mouse lungs.

FIG. 14 shows similar PKG1 and PKG2 mRNA expression in WT and Cav1^{-/-} mouse lungs.

FIG. 15 shows basal low levels of PKG-1 S-nitrosylation in mouse lungs.

FIG. 16 shows sequence alignment analysis demonstrating conserved tyrosine residues.

FIG. 17 shows L-NAME-treated Cav1^{-/-} mice had significantly fewer muscularized distal pulmonary arteries.

FIG. 18 shows normalization of PKG-mediated phosphorylation of VASP in Cav1^{-/-} lungs transfected with AdvPKG.

FIG. 19 shows eNOS-derived NO production in human lung tissue determined by a three-electrode system.

FIG. 20 shows normalized ERK signaling and gene expression in DKO lungs.

FIG. 21 shows impaired PKG kinase activity secondary to tyrosine nitration in Cav1^{-/-} lungs.

### DETAILED DESCRIPTION

The inventors have made the surprising discovery that there is an association between the molecular mechanisms derived from endothelial nitric oxide synthase (eNOS) and protein kinase G (PKG) activities, and pulmonary hypertension. eNOS and PKG have been identified as critical components in a system that regulates pulmonary vascular function. eNOS may regulate basal pulmonary vasomotor tone and its activity may be controlled by fatty acid modification, phosphorylation, as well as interaction with effector molecules. Caveolin-1 (Cav1) binding to eNOS may negatively regulate eNOS activity. Subjects having PH may have decreased expression of Cav1 in their lungs and/or endothelial cells. Subjects having PH may have enhanced or chronically active eNOS. eNOS activation may result in increased nitrative stress and/or oxidative stress. Nitrative and/or oxidative stress may result in tyrosine nitration of PKG. Tyrosine nitration of PKG may result in impaired PKG activity in lung vascular smooth muscle cells. Impaired PKG activity may result in one or more of vasoconstriction, pulmonary vascular change, and PH.

An agent that reduces or inhibits eNOS and/or nitrative stress-induced modification of PKG, therefore, may be useful for treating pulmonary hypertension. In addition, early detection of a PH-marker, such as PKG nitration, may allow treatment of the subject thereby delaying or preventing PH.

### 1. DEFINITIONS

The terminology used herein is for the purpose of describing particular embodiments only and is not intended to be limiting. As used in the specification and the appended claims, the singular forms “a,” “and” and “the” include plural references unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6,9, and 7.0 are explicitly contemplated.

a. Fragment

“Fragment” as used herein may mean a portion of a reference peptide or polypeptide or nucleic acid sequence.

b. Identical

“Identical” or “identity” as used herein in the context of two or more polypeptide or nucleotide sequences, may mean that the sequences have a specified percentage of residues or nucleotides that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

c. Label

“Label” or “detectable label” as used herein may mean a moiety capable of generating a signal that allows the direct or indirect quantitative or relative measurement of a molecule to which it is attached. The label may be a solid such as a microtiter plate, particle, microparticle, or microscope slide; an enzyme; an enzyme substrate; an enzyme inhibitor; coenzyme; enzyme precursor; apoenzyme; fluorescent substance; pigment; chemiluminescent compound; luminescent substance; coloring substance; magnetic substance; or a metal particle such as gold colloid; a radioactive substance such as ¹²⁵I, ¹³¹I, ³²P, ³H, ³⁵S, or ¹⁴C; a phosphorylated phenol derivative such as a nitrophenyl phosphate, luciferin derivative, or dioxetane derivative; or the like. The enzyme may be a dehydrogenase; an oxidoreductase such as a reductase or oxidase; a transferase that catalyzes the transfer of functional groups, such as an amino; carboxyl, methyl, acyl, or phosphate group; a hydrolase that may hydrolyzes a bond such as ester, glycoside, ether, or peptide bond; a lyases; an isomerase; or a ligase. The enzyme may also be conjugated to another enzyme.

The enzyme may be detected by enzymatic cycling. For example, when the detectable label is an alkaline phosphatase, a measurement may be made by observing the fluorescence or luminescence generated from a suitable substrate, such as an umbelliferone derivative. The umbelliferone derivative may comprise 4-methyl-umbellipheryl phosphate.

The fluorescent or chemiluminescent label may be a fluorescein isothiocyanate; a rhodamine derivative such as rhodamine β isothiocyanate or tetramethyl rhodamine isothiocyanate; a dancyl chloride (5-(dimethylamino)-1-naphtalenesulfonyl chloride); a dancyl fluoride; a fluorescamine (4-phenylspiro[furan-2(3H); 1ÿ-(3ÿH)-isobenzofuran]-3; 3ÿ-dione); a phycobiliprotein such as a phycocyanine or physoerythrin; an acridinium salt; a luminol compound such as lumiferin, luciferase, or aequorin; imidazoles; an oxalic acid ester; a chelate compound of rare earth elements such as europium (Eu), terbium (Tb) or samarium (Sm); or a coumarin derivative such as 7-amino-4-methylcoumarin.

The label may also be a hapten, such as adamantine, fluoroscein isothiocyanate, or carbazole. The hapten may allow the formation of an aggregate when contacted with a multi-valent antibody or (strep) avidin containing moiety. The hapten may also allow easy attachment of a molecule to which it is attached to a solid substrate.

The label may be detected by quantifying the level of a molecule attached to a detectable label, such as by use of electrodes; spectrophotometric measurement of color, light, or absorbance; or visual inspection.

d. Substantially Identical

“Substantially identical,” as used herein may mean that a first and second protein or nucleotide sequence are at least 50%-99% identical over a region of 8-100 or more amino acids nucleotides.

e. Therapeutically Effective Amount

“Therapeutically effective amount” as used herein may mean the amount of the subject agent or compound that will elicit the biological or medical response of a tissue, system, or subject that is being sought by the researcher, veterinarian, medical doctor or other clinician, and includes that amount of an agent or compound that, when administered, is sufficient to prevent development of, or alleviate to some extent, one or more of the symptoms of the condition or disorder being treated.

### 2. METHOD OF TREATMENT

Provided herein is a method of treating a subject diagnosed with PH or having a predisposition for PH. The subject may have a PH-marker. The method may comprise administering a PKG-effector agent to the subject. The PH may be a primary disease or a secondary disease. The PH may be pulmonary arterial hypertension or idiopathic pulmonary arterial hypertension. The PH may be secondary to an interstitial lung disease. The interstitial lung disease may be pulmonary fibrosis and/or scleroderma.

In any patient that carries a PH-marker, an assessment may be made as to whether the subject is an early disease subject, wherein PH has not occurred, or whether the subject has an increase in vasoconstriction, plexiform lesions, and/or thrombosis, for example. The assessment may indicate an appropriate course of preventative or maintenance treatment. The treatment therapy may be administered in different clinical settings during the life of a PH subject: (1) during early PH disease a subject may receive one or more PKG-effector agents to delay onset of one or more characteristics associated with PH, such as vasoconstriction, plexiform lesions, and/or thrombosis; (2) after a subject has been diagnosed as having one or more PH characteristics, one or more PKG-effector agents may be administered to slow any decline in pulmonary function and reduce frequency and morbidity of pulmonary exacerbations; and/or (3) during periodic exacerbations in pulmonary symptoms, PKG-effector agent regimens may be administered to relieve symptomotology and restore pulmonary function to baseline values.

Provided herein is a method of preventing or delaying onset of PH. An agent may be administered as part of a combination treatment with one or more other compounds. The subject may be undergoing treatment for another disease. The PH may be secondary to the other disease.

The treatment of a subject with an agent may be monitored by determining protein, mRNA, and/or transcriptional level of a gene. The gene may be a NOS gene. The NOS gene may be eNOS. The treatment of a subject with an agent may be monitored by determining the level of PKG nitration.

Depending on the level of protein, mRNA, transcriptional level of a gene, or level of PKG-nitration detected, the therapeutic regimen may be maintained or adjusted. The effectiveness of treating a subject with a PKG-effector agent may comprise (1) obtaining a preadministration sample from a subject prior to administration of the agent; (2) detecting the level or amount of a protein, RNA or DNA in the preadministration sample; (3) obtaining one or more post-administration samples from the subject; (4) detecting the level of expression or activity of the protein, RNA or DNA in the postadministration sample; (5) comparing the level of expression or activity of the protein, RNA or DNA in the preadministration sample with the corresponding protein, RNA, or DNA in the postadministration sample, respectively; and (6) altering the administration of the agent to the subject accordingly.

Cells of a subject may be obtained before and after administration of a therapeutic to detect the level of expression of genes other than the gene of interest to verify that the therapeutic does not increase or decrease the expression of genes that could be deleterious. Verification may be accomplished by transcriptional profiling. mRNA from cells exposed in vivo to a therapeutic and mRNA from the same type of cells that were not exposed to the agent may be reverse transcribed and hybridized to a chip containing DNA from many genes. The expression of genes in the treated cells may be compared against cells not treated with the agent.

Appropriate PKG-effector therapy may be essential steps in the management of PH. PH-effector agent selection for any given subject in any given setting may be based on periodic isolation and identification of PH-markers from samples from a subject.

a. Subject

The subject may be a human. The subject may be diagnosed with PH or have a predisposition for PH. The subject having PH or predisposed to PH may have a PH-marker. The pulmonary hypertension may result from nitration of one or more tyrosine residues of PKG. The nitration of PKG may result from an increase in nitrative and/or oxidative stress in the system in which PKG is present. The nitrative and/or oxidative stress may result from chronically active eNOS.

b. PKG-Effector Agent

The PKG-effector agent may be any compound that directly or indirectly decreases or inhibits tyrosine nitration of PKG, or enhances or increases PKG activity. The agent may decrease nitrative or oxidative stress in a cell. The agent may block preoxynitrate formation, thereby inhibiting tyrosine nitration of PKG. The agent may block formed preoxynitrate from nitrating PKG.

PKG may be PKG type I (PKG1) or type II (PKG2). The PKG may be mammalian. PKG may be nitrated at tyrosine residue 549 and/or tyrosine residue 345 and/or tyrosine residue 124 of full length PKG1 and/or PKG2.

The PKG-effector agent may be a peroxynitrate scavenger, a superoxide scavenger, a flavonoid, a NOS inhibitor, a PKG activator or enhancer, a NADPH oxidase inhibitor, a superoxide dismutase activator, a peroxidase activator, a catalase activator, an antioxidant, or combinations thereof.

(1) Peroxynitrate Scavenger

The peroxynitrate scavenger may be any compound that reacts directly with peroxynitrite anion or peroxynitrous acid to increase the rate of peroxynitrite decomposition in proportion to their concentration. The peroxynitrite may be any compound that scavenges secondary reactive species produced from reactions with peroxynitrite or peroxynitrous acid, thereby reducing reactive radical intermediates.

The peroxynitrate scavenger may be uric acid, a plant extracted proanthocyanidin, ascorbate, trolox, glutathione (GSH), Mn (III) tetrakis (4-benzoic acid) porphyrin (MnTBAP), flavonoid, ebselen, catchol (1,2-dihydroxybenzene), kaempferol, galangin, caffeic acid, o-coumaric acid, p-coumaric acid, gallic acid, and ferulic acid. The proanthocynidin may be extracted from an arborescent or herbaceous plant species.

(2) Superoxide Scavenger

The superoxide scavenger may be any compound or molecule that scavenges superoxide based on the reaction:

2O₂⁻+2H⁺→H₂O₂+O₂

The superoxide scavenger may be one or more of manganese (III) tetrakis (1-methyl-4-pyridyl) porphyrin pentachloride (MnTMPyP), 1-oxyl-2,2,6,6-tetramethyl-4-hydroxypiperidine (TEMPOL), and NAD(P)H:quinone oxidoreductase 1. The superoxide scavenger may be an antioxidant.

(3) Flavonoid

The flavonoid may be any plant phenolic having a flavan nucleus. The flavonoid may be found in fruits, vegetables, wines, teas and/or cocoa. The flavonoid may be one or more of quercetin, rutin, morin, acacetin, hispidulin, hesperidin, and/or naringin.

(4) NOS Inhibitor

The NOS inhibitor may be a selective, or specific, inhibitor or a non-selective inhibitor. The NOS inhibitor may be an eNOS inhibitor. The eNOS inhibitor may be selective or specific for eNOS. The eNOS inhibitor may decrease or inhibit eNOS function by interacting with eNOS protein thereby reducing production of eNOS-derived nitric oxide. The eNOS inhibitor may decrease the production of eNOS by downregulating expression of eNOS. The eNOS inhibitor may inhibit or decreasing expression of eNOS-encoded DNA or RNA.

The NOS inhibitor may be N omega-nitro-L-arginine, N omega-monomethyl-L-arginine, 1-N^{G} monomethyl arginine (1-NMMA), ARL 17477, a caveolin-1 peptide, and KLYP956. The caveolin-1 peptide may be a mimetic. The caveolin peptide may comprise the sequence DGIWKASFTTFTVTKYWFYR (SEQ ID NO. 1). The caveolin peptide sequence may consist of SEQ ID NO:1. The caveolin peptide may be substantially identical to SEQ ID NO:1. SEQ ID NO:1 may bind to eNOS.

(5) PKG Activator or Enhancer

The PKG activator or enhancer may be any compound or protein that increases PKG activity. The activator or enhancer may directly bind to PKG. The activator or enhancer may increase expression of PKG encoded DNA or RNA.

The PKG activator or enhancer may be one or more of phosphatidylinositol-3,4,5-trisphosphate (PtdIns(3,4,5)P3), cyclic guanosine 3′, 5′-monophosphate (cGMP), 8-pCPT-cGMP (cGMP derivative), and/or a cGMP phosphodiesterase inhibitor. The cGMP phosphodiesterase inhibitor may be sulindac sulfone, sildenafil, tadalafil, and/or OSI-461.

(6) NADPH Oxidase Inhibitor

The NADPH oxidase inhibitor may be any compound or protein that decreases or halts NADPH expression or activity. The inhibitor may directly bind to NADPH oxidase. The inhibitor may decrease or halt expression of NADPH oxidase encoded DNA or RNA. The NADPH oxidase inhibitor may be apocynin and/or diphenylene iodonium. The NADPH oxidase inhibitor may be an antioxidant. The NADPH oxidase inhibitor may ultimately prevent or inhibit the formation of free radicals, for example reactive O₂ species, thereby making it an antioxidation component.

(7) Superoxide Dismutase Activator

The superoxide dismutase activator may be any compound or protein that that increases or enhances superoxide dismutase activity or expression. The activator may directly bind to superoxide dismutase. The activator or enhancer may increase expression of superoxide dismutase encoded DNA or RNA.

The superoxide dismutase activator may be one or more of a lipid peroxide, reduced glutathione, and/or 17β-estradiol. The activator may be an antioxidant. The superoxide dismutase activator may ultimately prevent or inhibit the formation of free radicals, for example reactive O₂ species, thereby making it an antioxidation component.

(8) Peroxidase Activator

The peroxidase activator may be any compound or protein that increases or enhances peroxidase activity or expression. The activator may directly bind to peroxidase. The activator may enhance or increase expression of peroxidase encoded DNA or RNA.

The peroxidase activator may be one or more of iron, copper, melatonin, NAC, and/or 4-hydrobenzoic acid. The peroxidase activator may be an antioxidant. The peroxidase activator may ultimately prevent or inhibit the formation of free radicals, for example reactive O₂ species, thereby making it an antioxidation component.

(9) Catalase Activator

The catalase activator may be any compound or protein that increases or enhances catalase activity or expression. The activator may directly bind to catalase. The activator may enhance or increase expression of catalase encoded DNA or RNA.

The catalase activator may be an oxidized linoliec acid. The oxidized linoliec acid may be one or more of 13-hydroperoxy-9,11-octadecadienoic acid (13-HPODE), 13-hydroxy-9,11-octadecadienoic acid (13-HODE), hydrogen peroxide, and oxidized low density lipoprotein (LDL). The catalase activator may be an antioxidant. The catalase activator may ultimately prevent or inhibit the formation of free radicals, for example reactive O₂ species, thereby making it an antioxidation component.

c. Compound for Combination Treatment

The method of treating PH may further comprise the administration of one or more compounds that inhibit phosphodiesterase activity and/or have endothelin receptor antagonist activity. The one or more compounds may be administered to the subject before, after, or at the same time as administering the PKG-effector agent.

The compound having phosphodiesterase activity may be a phosphodiesterase type-5 (PDE5). The PDE5 may be sildenafil, avanafil, tadalafil, acetildenafil, cGMP specific phosphodiesterase type-5, udenafil, vardenafil, and any combination thereof.

The compound having endothelin receptor antagonist activity may be one or more of atrasentan, bosentan, sitaxsentan, and ambrisenten.

d. Formulations and Administration

The PKG-effector agent and/or the compound for combination treatment may be formulated for parenteral administration by injection, e.g., by bolus injection or continuous infusion. Formulations for injection may be presented in unit dosage form, e.g., in ampules or in multi-dose containers, with an added preservative. The PKG-effector agent and/or the compound for combination treatment may take such a form as a suspension, solution, or emulsion in oily or aqueous vehicles, and may contain formulating agents such as suspending, stabilizing and/or dispersing agents. PKG-effector agent and/or the compound for combination treatment preparations for oral administration may be suitably formulated to give controlled release of the PKG-effector agent and/or the compound for combination treatment. For buccal administration, the PKG-effector agent and/or the compound for combination treatment may take the form of tablets or lozenges formulated in conventional manner. For administration by inhalation, the PKG-effector agent and/or the compound for combination treatment for use according to the present invention may be delivered in the form of an aerosol spray presentation from pressurized packs or a nebuliser, with the use of a suitable propellant. In the case of a pressurized aerosol, the dosage unit may be determined by providing a valve to deliver a metered amount.

In general, the PKG-effector agent and/or the compound for combination treatment of this invention will be administered in a therapeutically effective amount by any of the accepted modes of administration for agents and compounds that serve similar utilities. The actual amount of the PKG-effector agent and/or the compound for combination treatment of this invention will depend upon numerous factors such as the severity of the disease to be treated, the age and relative health of the subject, the potency of the compound used, the route and form of administration, and other factors. The PKG-effector agent and/or the compound for combination treatment can be administered more than once a day, preferably once or twice a day. Therapeutically effective amounts of an PKG-effector agent and/or the compound for combination treatment may range from approximately 0.05 mg to 10 g per kilogram body weight of the subject per day.

### 3. METHOD OF DIAGNOSIS

The detection of a PH-marker in a sample from a subject may be indicative of the subject having PH or having a predisposition for PH. The method may detect PKG nitration in a subject's lung. The method may detect PKG nitration in a sample taken from the subject. The sample may be a biopsy. The sample or biopsy may be a lung sample or biopsy. The method may use antibodies to PKG. The PKG may be nitrated. Nitrated PKG may be nitrated PKG-1α, PKG-1β, PKG-2, or peptides thereof. The PKG or PKG peptide may be human. The nitrated PKG or PKG peptide may be nitrated at tyrosine residue 549 and/or 345 and/or 124. The nitrated residues may correspond to tyrosine 549, 345, and/or 124 of PKG-1α. The detection of a nitrated PKG in a subject sample may be compared to a control sample. The presence of nitrated PKG in a subject sample may be indicative of PH. An increased level of PKG nitration in the subject sample as compared to a normal control sample may be indicative of the subject having PH or having a predisposition for PH. The pulmonary hypertension may be idiopathic pulmonary hypertension.

a. Sample

The sample may be any cell type, tissue, or bodily fluid from the subject. Such cell types, tissues, and fluid may include sections of tissues such as biopsy and autopsy samples, frozen sections taken for histologic purposes, blood, plasma, serum, sputum, stool, tears, mucus, saliva, hair, and skin. Cell types and tissues may also include lung tissue or cells, lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from a subject, but can also be accomplished by using previously isolated cells (e.g., isolated by another person, at another time, and/or for another purpose). Archival tissues, such as those having treatment or outcome history, may also be used.

b. PH-Marker

The PH-marker may be a protein marker. The marker may be PKG. PKG may be PKG type I (PKG1) or type II (PKG2). The PKG may be mammalian. The PKG may be nitrated at tyrosine residue 549 and/or tyrosine residue 345 and/or tyrosine residue 124 of full length PKG1 and/or PKG2.

c. Detection

The PH-marker may be detected in a sample derived from the patient. Many methods are available for detecting a marker in a subject or in a sample taken from the subject. These methods include immunological methods, which may be used to detect such proteins on whole cells. Expression of a PH-marker may be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In a preferred embodiment, the presence or expression of a PH-marker may be assessed using an antibody. The antibody may be labeled (e.g. a radio-labeled, chromophore-labeled, fluorophore-labeled, or enzyme-labeled antibody). The antibody may be an antibody derivative (e.g. an antibody conjugated with a substrate or with the protein or ligand of a protein-ligand pair (e.g. biotin-streptavidin)), or an antibody fragment (e.g. a single-chain antibody, an isolated antibody hypervariable domain, etc.) which binds specifically with the PH-marker.

### 4. METHOD OF MONITORING PH

Also provided herein is a method of monitoring a subject for PH. The subject may have been determined to have a predisposition for PH. The subject may already have a primary pulmonary disease, such as pulmonary fibrosis. It may be desirable to measure the effects of treatment on PH by treating the patient using a method comprising monitoring PKG nitration. Monitoring for PKG nitration, or progression of PKG nitration, may include any assay to detect PKG nitration.

### 5. KIT

Provided herein is a kit, which may be used for diagnosing, monitoring, or treating PH. The kit may comprise a sample collecting means. The kit may also comprise a means for determining a PH-marker, a PH-marker for use as a positive control, and/or a PH-marker detection means. The detection means may include substrates, such as filter paper, and protein purification reagents. Marker detection means may also be included in the kit. Such means may include primary and secondary antibodies and one or more buffers. The kit may also comprise a control sample. The control sample may not comprise a PH-marker.

The kit may also comprise one or more containers, such as vials or bottles, with each container containing a separate reagent. The kit may further comprise written instructions, which may describe how to perform or interpret an assay or method described herein.

The present invention has multiple aspects, illustrated by the following non-limiting examples.

### EXAMPLES

### Example 1

### Generation of DKO Mice

Throughout the Examples, data are presented as mean±SD. Statistical significance of differences between group means was determined using an unpaired two-tailed Student's t-test; P<0.05 was considered significant.

To generate the DKO mice, eNOS^{-/-} mice (The Jackson Laboratory) were bred into the background of Cav1^{-/-} mice (The Jackson Laboratory) to produce Cav1+/−/eNOS+/−double heterozygote (F1). F1 pups were then mated to generate WT, Cav1^{-/-}, eNOS^{-/-}, and Cav1^{-/-}/eNOS^{-/-} (DKO) mice. All mice were bred and maintained in the University of Illinois according to NIH guidelines. Approval for animal care and use for these experiments was granted by the institutional Animal Care and Use Committee.

### Example 2

### Molecular Analysis

RNA was isolated using an RNeasy Mini kit including DNase I digestion (Qiagen) and one-step QRT-PCR analyses were performed in ABI Prism 7000 Sequence Detection System (Applied Biosystems) with QuantiTect SYBR Green RT-PCR kit (Qiagen). The following primer sets were used for analyses: mouse p21Cip1, 5′-GACAAGAGGCCCAGTACTTCCT-3′ (SEQ ID NO:2) and 5′-CAATCTGCGCTTGGAGTGATA-3′ (SEQ ID NO:3); mouse IGF-1,5′-ACAGGCTATGGCTCCAGCAT-3′ (SEQ ID NO:4) and 5′-GCTCCGGAAGCAACACTCAT-3′ (SEQ ID NO:5); and mouse VEGF-A, 5′-TCCAAGATCCGCAGACGTGTAA-3′ (SEQ ID NO:6) and 5′-TGGCTTGTCACATCTGCAAGTAC-3′ (SEQ ID NO:7). Mouse cyclophilin primer set not shown. All gene expression was normalized to cyclophilin as an internal control.

Western blot analyses were performed using anti-Cav1 (1:1000, Santa Cruz Biotechnology), anti-eNOS (1:1000, Upstate Biotechnology), and anti-PKG-I (1 mg/ml, a generous gift from Dr. X-P. Du at Department of Pharmacology, University of Illinois at Chicago). The same blots were reprobed with either anti-a-actin (1:400, Santa Cruz Biotechnology) or anti-GAPDH (1:2000, Santa Cruz Biotechnology) as a loading control. To detect PKG-I tyrosine nitration, protein lysates from either mouse lungs or primary cultures of human pulmonary artery smooth muscle cells were immunoprecipitated overnight with anti-PKG-I and then probed with anti-nitrotyrosine (1:2500, Cayman Chemical).

### Example 3

### NO Measurement

NO measurements were performed using a three-electrode system. Briefly, lungs were cut into 1 mm thick slices and preincubated in L-arginine-free HBSS at 370 C for 1 h. With the aid of a micromanipulator, the NO sensor was carefully placed on the surface of the lung slice and the baseline was recorded. To determine the lung eNOS activity, the samples were subsequently incubated in a bath containing HBSS, 1 mM L-arginine, 2 mM iNOS inhibitor (1400 W) for 20 min. 1 mM calcium ionophore (A23187) was added and NO release was recorded for 20 s. NO production was measured as area under the curve during the 20-min period.

### Example 4

### In Vivo Measurement

RVSP was determined as follows. Briefly, following anesthesia, mice were prepared for catheterization. A 1.4 F pressure transducer catheter (Millar Instruments) was carefully inserted via the right external jugular vein into the right ventricle to obtain measurements of RVSP using the Acknowledge software (Biopac Systems, Inc.). To determine lung vascular resistance, mice were anesthetized and prepared for perfusion. Pulmonary arterial (Ppa) and venous (Pv) pressures were monitored continuously through the pulmonary arterial and left atrial cannula, which were connected to a pressure transducer. After a period of 20 min during which isogravimetric conditions were attained, the flow was stepped cumulatively from 2 to 3.5, and then to 5 mL/min. Ppa was measured at steady-state and was plotted against the flow, which was measured by weighing the effluent collected over a one-minute period. The pulmonary vascular resistance was derived as the slope of the pressure flow curve.

### Example 5

### PKG Kinase Assay

In vitro activity of PKG was determined by measuring the transfer of the [g-32P] phosphate group of ATP to the specific PKG substrate, BPDEtide (Calbiochem) in the absence or presence of exogenous 2.5 mM cGMP. The assay was carried out in a total volume of 50 ml containing 150 mM BPDEtide, 10 mM HEPES, 35 mM b-glycerophosphate, 4 mM magnesium acetate, 5 mM PKI (a synthetic protein kinase A inhibitor, Calbiochem), 0.5 mM EDTA, 200 mM ATP, and 2 mCi of [g-32P]ATP (specific activity 3,000 Ci/mmol, GE Amersham). The mixture was incubated at 300 C for 12 min and terminated by spotting 40 ml aliquots of mixture on phosphocellulose papers (P81, Whatman). The papers were then washed and counted in a liquid scintillation counter. PKG activity is expressed as picmoles of 32P incorporated into PKG substrate per minute per milligram protein.

### Example 6

### Histological Analysis and Immunohistochemistry

Following PBS perfusion, the lung tissues were fixed for 5 min by instillation of 10% PBS-buffered formalin through trachea catheterization at a transpulmonary pressure of 15 cm H₂O. After tracheal ligation, harvested lungs were fixed with 10% PBS-buffered formalin overnight at 40 C with agitation. After paraffin processing, the tissues were cut into semithin 4 to 5 mm thick, and stained with either H & E for histological analysis or an antibody against nitrotyrosine (Upstate Biotechnology) for immunohistochemistry. Immunostaining was developed with a Vectastain ABC kit (Vector Laboratories).

### Example 7

### Genetic Deletion of eNOS in Cav1^{-/-} Mice Prevents PH

To generate a mouse model with genetic deletions of both Cav1 and eNOS, eNOS^{-/-} mice 24 were mated into the background of Cav1^{-/-} mice 25. DKO mice were born normally and were indistinguishable from wild-type (WT) littermates. We observed that 85% (n=200) of DKO mice survived as long as WT mice (up to 18 month). To eliminate any background effects from either eNOS^{-/-} or Cav1^{-/-} line on the observed phenotype of DKO mice, F4 or higher generations were used for these studies.

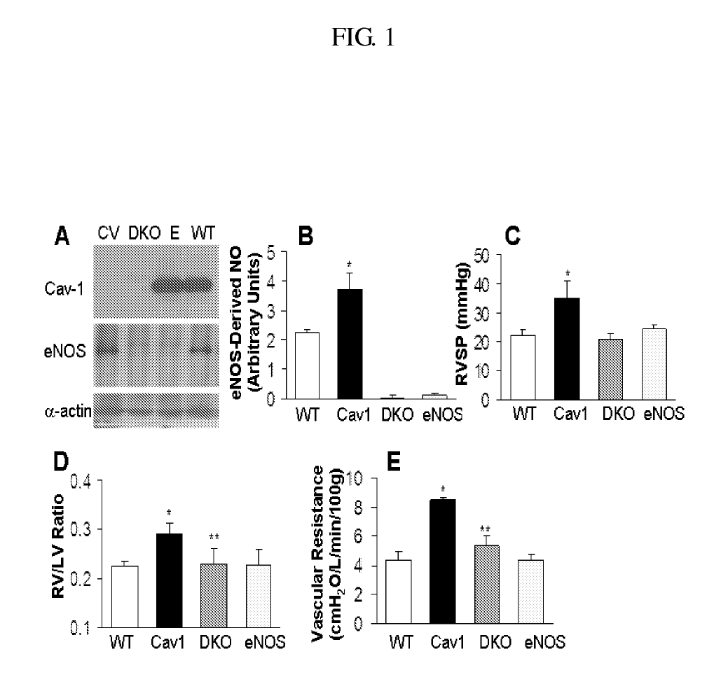

FIG. 1A shows a Western blot analysis of Cav1 and eNOS expression in lungs from 2 month old mice. Lung lysate (25 mg per lane) was loaded and immunoblotted with antibodies against mouse Cav1, eNOS, and a-actin (loading control), respectively. Neither Cav1 nor eNOS was detected in DKO lungs. The experiment was repeated three times with similar data. CV, Cav1^{-/-}; E, eNOS^{-/-}. As shown in FIG. 1A, Cav1^{-/-} lungs expressed the same amount of eNOS as −/− WT. However, eNOS-derived NO in Cav1^{-/-} lungs was 2-fold greater than in WT lungs (FIG. 1B), indicating activation of eNOS secondary to loss of Cav1 inhibition in vivo. There was little eNOS-derived NO produced in either eNOS^{-/-} lungs or DKO lungs (FIG. 1B). FIG. 1B shows a quantitative analysis of eNOS-derived NO in mouse lungs. Data are shown as mean±SD. *, P=0.001 versus WT (n=4-6). Cav1, Cav1^{-/-}; eNOS, eNOS^{-/-}. Cav1^{-/-} lungs produced greater eNOS-derived NO whereas NO production was markedly reduced in DKO mice. Similar levels of expression of both iNOS and nNOS were detected in lungs from the four genetic backgrounds (data not shown). The amount of iNOS-derived NO from Cav1^{-/-} lungs was the same as WT lungs (data not shown). Therefore, previously identified increase in plasma NO levels in Cav1^{-/-} mice 20 is primarily the result of constitutive activation of eNOS.

To determine physiological consequences of chronic activation of eNOS in Cav1^{-/-} lungs, right ventricular systolic pressure (RVSP) was measured as indicative of pulmonary arterial systolic pressure. FIG. 1C shows Normalized RVSP in DKO mice. RVSP was measured in age- and gender-matched mice (9 month). Data are expressed as mean±SD. *, P=0.002 Cav1 versus WT and P=0.003 Cav1 versus DKO (n=6-8). As shown in FIG. 1C, Cav1^{-/-} mice exhibited significantly increased RVSP compared to WT. However, RVSP in DKO mice was the same as WT mice. Right/left ventricle (RV/LV) weight ratio, an index for right ventricular hypertrophy, was normal in DKO mice in contrast to Cav1^{-/-} mice (FIG. 1D). FIG. 1D shows the Restored RV/LV weight ratio in DKO mice. Right and left ventricles including the septum were dissected free of connective tissues and weighed from age- and gender-matched mice. Data are mean±SD. *, P=0.0015 Cav1 versus WT (n=5-7); **, P=0.005 DKO versus Cav1 (n=6-8).

FIG. 1E shows Normalization of pulmonary vascular resistance in DKO lungs. Data are expressed as mean±SD. *, P=0.001 Cav1 versus WT and P=0.013 Cav1 versus DKO; **, P>0.5 DKO versus WT (n=4-6). As shown in FIG. 1E, PVR in DKO mice was reduced to the similar level as in WT lungs. These data suggest that PH in Cav1^{-/-} mice is the result of chronic activation of eNOS.

### Example 8

### DKO Lungs Exhibit Normal Pulmonary Vasculature and Expression of p21Cip1

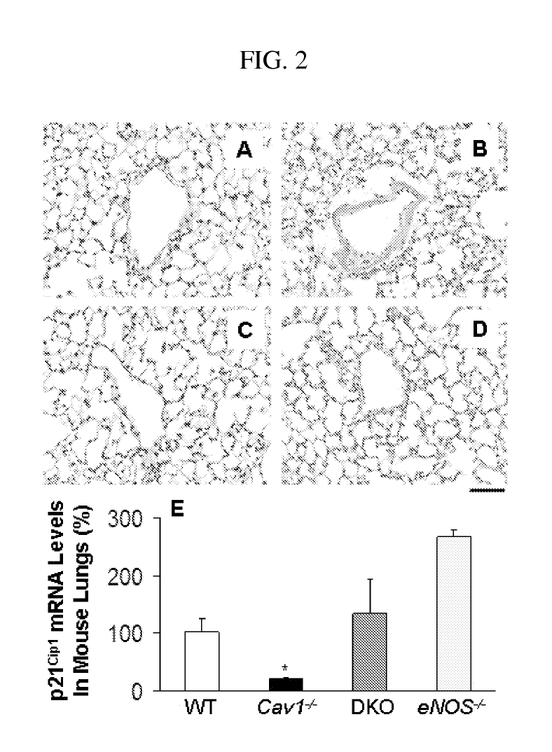

Studies have demonstrated severe lung structural abnormalities in Cav1^{-/-} mice such as hypercellularity and thickened alveolar septa 18,20,25. To address whether genetic ablation of eNOS prevents the lung pathology seen in Cav1^{-/-} lungs, we processed lungs for histological studies. FIG. 2A-D shows representative micrographs of H & E staining of lung sections from age- and gender-matched WT (A), Cav1^{-/-} (B), DKO (C), and eNOS^{-/-} mice (D). Cav1^{-/-} lungs exhibited hyper-cellularity and medial thickening which were prevented in DKO mouse lungs. Scale bar, 50 mm. (E) Restored expression of p21Cip1 in DKO lungs. Lungs were collected from 2 month old male mice and RNA was isolated for QRT-PCR analysis. p21Cip1 mRNA levels were normalized to cyclophilin. Data are expressed as mean±SD (n=3-4). *, P<0.001 versus WT or DKO.

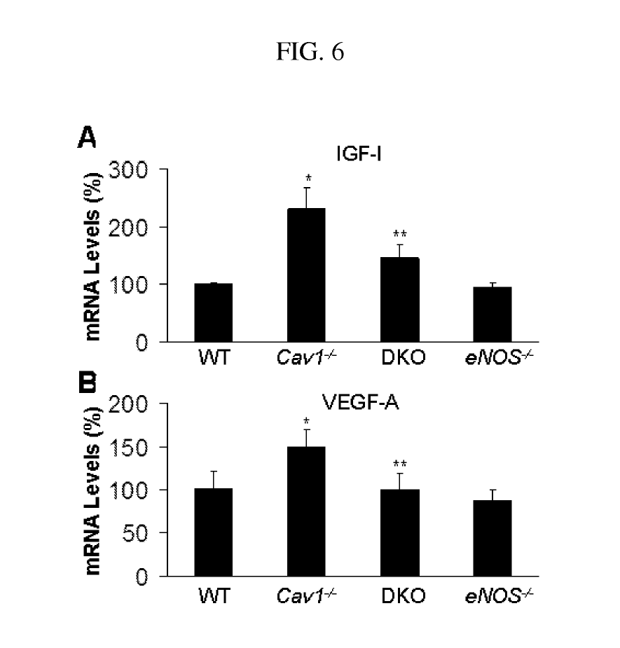

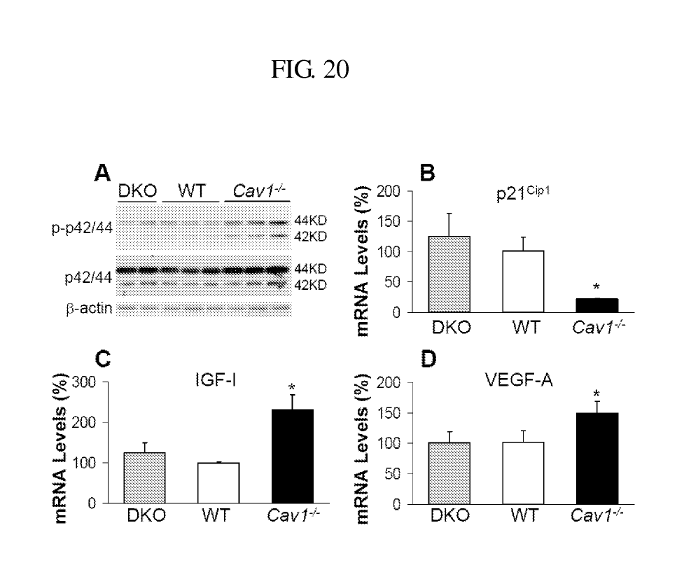

As shown in FIG. 2, DKO lungs exhibited normal alveolar-capillary structure and vessel wall thickness (FIG. 2C) in contrast to Cav1^{-/-} lungs (FIG. 2B). To address the molecular basis of the hyperplasia seen in Cav1^{-/-} lungs, we also examined expression of a set of genes regulating cell cycle progression. Quantitative reverse transcription-polymerase chain reaction (QRT-PCR) analysis showed that the mRNA level of p21Cip1, a cyclin-dependent kinase inhibitor, was decreased 5-fold in Cav1^{-/-} lungs compared to WT lungs, whereas p21Cip1 expression in DKO lungs was restored (FIG. 2E). eNOS^{-/-} lungs expressed greater than 2-fold p21Cip1 than WT lungs, suggesting an eNOS-dependent mechanism of transcriptional regulation of p21Cip1. Previous cell culture studies in contrast showed that exogenous NO induced the expression of p21Cip1, thereby blocking cell proliferation 26. Interestingly, expression of several growth factors such as insulin-like growth factor 1 (IGF-I) and vascular endothelial growth factor-A (VEGF-A) was increased in Cav1^{-/-} lungs, whereas they were normal in DKO lungs (FIGS. 6 and 20). With regard to FIG. 6, total RNA was isolated from lungs collected from 2 month old mice and mRNA levels of IGF-I (A) and VEGF-A (B) were analyzed with quantitative SYBR Green assay. mRNA levels of cyclophilin were used for normalization. Data are expressed as mean±SD (n=3-5). *, P<0.01 versus WT; **, P<0.05 versus Cav1^{-/-}. With regard to FIG. 20B-D, quantitative analyses were performed of gene expression in mouse lungs. Total RNA was isolated from lungs collected from 2-month old mice and mRNA levels of p21Cip1 (B), IGF-I (C) and VEGF-A (D) were analyzed with quantitative SYBR Green RT-PCR assay. mRNA levels of cyclophilin were used for normalization. Data are expressed as mean±SD (n=3-5). *, P<0.01 versus either WT or DKO. Expression of p21Cip1 and growth factors IGF-I and VEGF-A was normalized in DKO lungs.

In view of the foregoing, downregulation of p21Cip1 in Cav1^{-/-} lungs may be the result of increased expression of these growth factors in Cav1^{-/-} lungs.

### Example 9

### Chronic eNOS Activation Secondary to Cav1 Deficiency Induces Peroxynitrite Formation and PKG Dysfunction Through Tyrosine Nitration

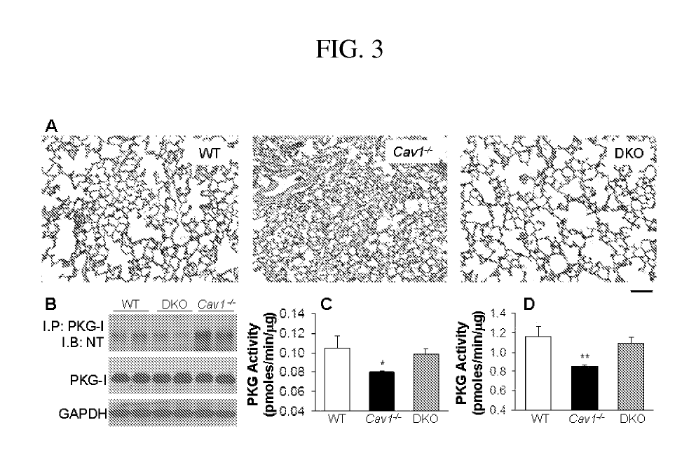

NO present in high concentration reacts with superoxide to form the damaging reactive nitrogen species peroxynitrite 27, which modifies proteins through tyrosine nitration. Using immunostaining of nitrotyrosine generated by tyrosine nitration as a measure of formation of peroxynitrite, Cav1^{-/-} lungs demonstrated prominent nitrotyrosine immunostaining whereas DKO lungs similar to WT lungs exhibited little nitrotyrosine immunostaining (FIG. 3A). FIG. 3A shows detection of peroxynitrite in lung sections by immunohistochemistry using anti-nitrotyrosine antibody. Representative micrographs from 4-6 animals per group are shown. Scale bar, 50 mm. Peroxynitrite was only detectable in Cav1^{-/-} lungs (Red).

We examined tyrosine nitration of PKG in Cav1^{-/-} lungs since PKG is the downstream target of NO signaling and its activation regulates vasorelaxation. FIG. 3B shows Prominent tyrosine nitration of PKG was detected in lung lysates from Cav1^{-/-} mice. Lung lysates (100 mg) from WT, DKO and Cav1^{-/-} mice, respectively, were immunoprecipitated with 5 mg of polyclonal antibody against human PKG-I overnight and PKG tyrosine nitration was detected with polyclonal antibody against nitrotyrosine (NT) by Western blotting. I.P, immunoprecipitation; I.B, immunoblot. Protein levels of PKG-I in lung lysates (20 mg per lane) were also determined directly by Western blotting using the same polyclonal antibody against human PKG-I. The same blot was reprobed with an antibody against mouse glyceraldehyde-3-phosphate dehydrogenase (GAPDH) used as a loading control. There were no differences in protein levels of PKG-I in lungs of WT, DKO, and Cav1^{-/-}. As shown in FIG. 3B, we observed a marked increase in nitrotyrosine-modified PKG-I in Cav1^{-/-} lung lysates compared to DKO and WT lungs. However, the same amount of PKG-I protein was expressed in lungs of Cav1^{-/-}, WT, and DKO. QRT-PCR demonstrated similar mRNA levels of both PKG-I and II in mouse lungs (data not shown).

In FIG. 3C-D, In vitro PKG activity in lung lysates at basal (C) or with addition of 2.5 μM of cGMP (D). Data are expressed as mean±SD (n=4-5). *, P=0.021 Cav1^{-/-} versus WT, and P=0.025 Cav1^{-/-} versus DKO; **, P=0.004 Cav1^{-/-} versus WT, and P=0.014 Cav1^{-/-} versus DKO. PKG kinase activity was determined using an in vitro kinase assay. PKG kinase activity in Cav1^{-/-} lungs was decreased 30% compared to either WT or DKO lungs at either basal condition (FIG. 3C) or with addition of 2.5 mM of cGMP (FIG. 3D). To investigate whether impaired cGMP production was involved in PKG dysfunction in Cav1^{-/-} lungs, cGMP levels in plasma and lung lysates were determined by ELISA. We observed no significant differences in cGMP levels of plasma and lung lysates of WT, Cav1^{-/-}, and DKO mice (data not shown).

### Example 10

### SIN-1 Causes PKG-I Tyrosine Nitration and Decreased Kinase Activity in Cultured Smooth Muscle Cells

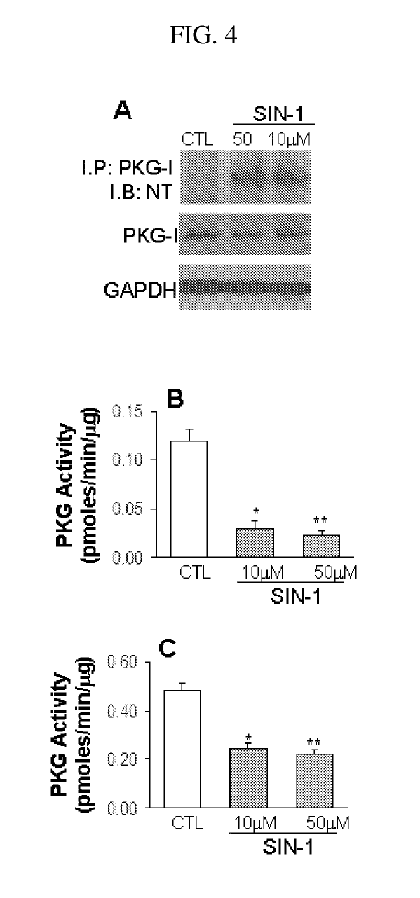

To test the hypothesis that peroxynitrite modifies PKG through tyrosine nitration thereby impairing kinase activity, we treated subconfluent human pulmonary artery smooth muscle cells with 3-morpholinosydnonimine (SIN-1), a donor of superoxide and NO, which spontaneously forms peroxynitrite at the two concentrations used. FIG. 4 shows (A) subconfluent primary cultures that were treated with SIN-1 at indicated doses for 30 min. Each cell lysate (50 mg) was used for immunoprecipitation with an anti-PKG-I antibody and tyrosine nitration of PKG-I was detected by Western blot with anti-nitrotyrosine antibody (NT). Each lysate (15 mg) was used for direct immunoblotting with anti-PKG-I and anti-GAPDH, respectively. CTL, control. FIG. 4B-C shows that SIN-1 treatment resulted in significant decrease of PKG activity either basally (B) or after addition of 2.5 mM of cGMP (C). Data are shown as mean±SD (n=3). *, P<0.001 versus CTL; **, P<0.001 versus CTL.

More specifically, in FIG. 4A, SIN-1 concentration as low as 10 mM caused significant PKG tyrosine nitration following 30 min treatment. Also SIN-1 treatment significantly decreased PKG kinase activity in smooth muscle cells (FIGS. 4B and 4C). SIN-1 treatment at 0.1 mM produced only minimal PKG tyrosine nitration and did not decrease PKG kinase activity (data not shown). Thus, production of eNOS-derived NO in Cav1^{-/-} lungs resulted in formation of peroxynitrite which impaired PKG kinase activity through tyrosine nitration, and thereby induced vasoconstriction instead of vasorelaxation.

### Example 11

### Pharmacological Inhibition of eNOS in Cav1^{-/-} Mice Reverses Pulmonary Defects Responsible for PH

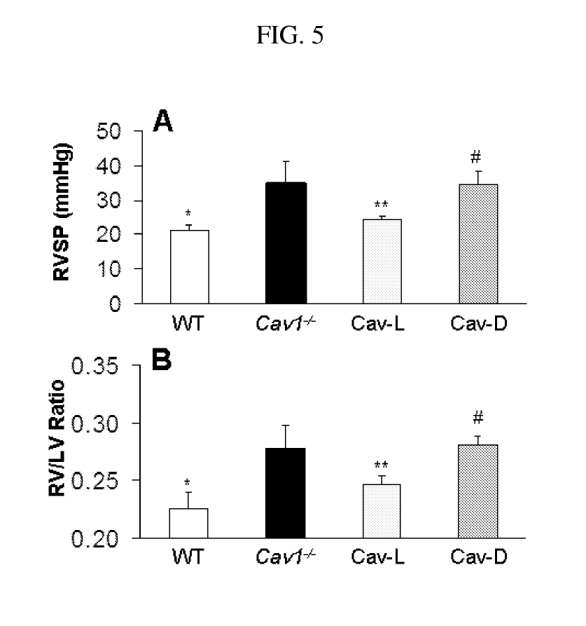

To determine whether pharmacological inhibition of eNOS in Cav1^{-/-} mice can reverse PH, we administered N-Nitro-L-Arginine Methyl Ester (L-NAME), a NOS inhibitor, to Cav1^{-/-} mice following onset of PH. In this treatment protocol, 8 month old Cav1^{-/-} mice received either L-NAME or its inactive analog D-NAME in drinking water (1 mg/ml) for 5 wk. In FIG. 5A, RVSP was decreased in Cav1^{-/-} mice following 5 wk treatment of L-NAME. Cav1^{-/-} male mice (8 month old) had ad libitum water (Cav1^{-/-}, control) or water with 1 mg/ml of L-NAME (Cav-L) or its inactive analog D-NAME (Cav-D) for 5 wk. Age- and gender-matched WT littermates were also included as a control. L-NAME treatment resulted in significant decrease of RVSP in Cav1^{-/-} mice whereas D-NAME treatment had no effect. Data are expressed as mean±SD (n=5-7). *, P=0.023 WT versus Cav1^{-/-};**, P=0.015 Cav-L versus Cav1^{-/-}; #, P>0.5 Cav-D versus Cav1^{-/-}. (B) L-NAME-treated Cav1^{-/-} mice exhibited significantly decreased RV/LV ratio. Data are expressed as mean±SD (n=5-7). *, P=0.002 WT versus Cav1^{-/-}; **, P=0.013 Cav-L versus Cav1^{-/-}; #, P=0.8 Cav-D versus Cav1^{-/-}.

More specifically, L-NAME treatment substantially reduced RVSP whereas D-NAME had no effect (FIG. 5A). RV/LV ratio was also significant reduced following 5-wk L-NAME treatment period (FIG. 5B) whereas LV/BW ratio did not change (data not shown). Histological examination showed normal lung morphology and reduced vascular remodeling. Thus, chronic overproduction of eNOS-derived NO plays a critical role in the pathogenesis of pulmonary vascular remodeling and PH seen in Cav1^{-/-} mice.

### Example 12

### General Methods

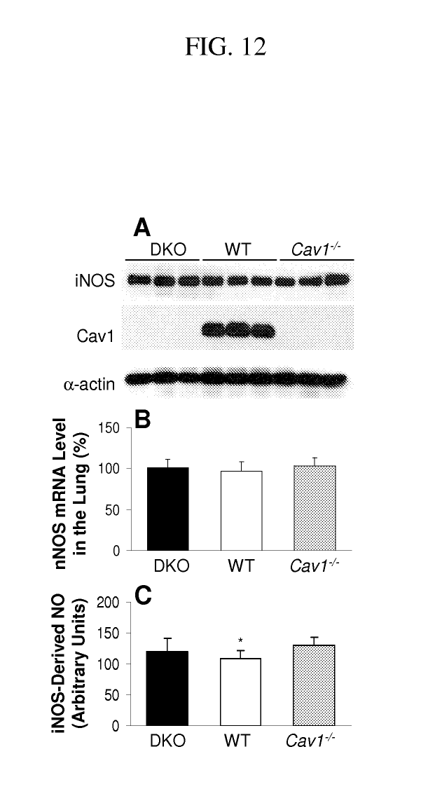

The following primer sets were used for analyses shown in FIGS. 12-19: mouse p21Cip1, 5′-GACAAGAGGCCCAGTACTTCCT-3′ (SEQ ID NO:2) and 5′-CAATCTGCGCTTGGAGTGATA-3′ (SEQ ID NO:3); mouse IGF-1,5′-ACAGGCTATGGCTCCAGCAT-3′ (SEQ ID NO:4), and 5′-GCTCCGGAAGCAACACTCAT-3′ (SEQ ID NO:5); mouse VEGF-A, 5′-TCCAAGATCCGCAGACGTGTAA-3′ (SEQ ID NO:6), and 5′-TGGCTTGTCACATCTGCAAGTAC-3′ (SEQ ID NO:7); mouse nNOS, 5′-ACCGAATACAGGCTGACGATGT-3′ (SEQ ID NO:8) and 5′-GCACGGATTCATTCCTTTGTGT-3′ (SEQ ID NO:9); mouse PKG-1,5′-CTGTCACAGATCCAGGAGATTG-3′ (SEQ ID NO:10) and 5′-ATCGCCTTCCTTGATGATGCAG-3′ (SEQ ID NO:11); and mouse PKG-2,5′-TTCAGTGTGGATTTCTGGTCCC-3′ (SEQ ID NO:12) and 5′-GTCATCATTTGGTCTATCCCAG-3′ (SEQ ID NO:13). All gene expression was normalized to cyclophilin as an internal control.

sGC Enzyme Activity and Lung cGMP Measurement. sGC enzyme activity was measured as previously described by Mittal. Briefly, lung tissue was homogenized in buffer containing 50 mM Tris.HCl (pH 7.5), 1 mM EDTA, 1 mM dithiothreitol, 2 mM phenylmethyl sulfonyl fluoride, 0.5 mM 3-isobutyl-1-methylxanthine, and protease inhibitor cocktail (Sigma-Aldrich). Extracts were centrifuged at 100,000 g for 1 h at 40 C. Supernatants (50 μg in 30 μl lysis buffer) were incubated for 10 min at 37° C. in a reaction mixture containing 50 mM Tris.HCl (pH 7.5), 5 mM MgCl₂, 0.5 mM 3-isobutyl-1-methylxanthine, 7.5 mM creatine phosphate, 0.2 mg/ml creatine phosphokinase, and 1 mM GTP with (Basal) or without 1 mM sodium nitroprusside. The total reaction volume was 100 μl The reaction was terminated by 0.1 N HCl. The cGMP in the reaction mixture following dilution with assay buffer (1:5 dilutions for the basal reaction mixture and 1:50 dilution for the sodium nitroprusside-stimulated reaction mixture) was measured using a commercial cGMP enzyme immunoassay kit (GE Healthcare) following manufacture's instruction. sGC activity is expressed as picomoles of cGMP produced per min per milligram of lung extract supernatant. To determine the lung cGMP levels, mouse lung tissue was homogenized in cold 6% (w/v) trichloroacetic acid and supernatant was collected following centrifugation at 2000 g for 15 min at 4° C. Following 4 washing of the supernatant with 5 volumes of water saturated diethyl ether, the aqueous extract of cGMP was lyophilized and resuspended in 220 μl of assay buffer. cGMP levels were measured with the same cGMP enzyme immunoassay kit following manufacture's instruction. Mouse lung cGMP levels were expressed as pmoles/g wet lung.

In Vitro PKG Kinase Assay. Mouse lung tissue was homogenized in 800 μl lysis buffer containing 10 mM HEPES, 0.5 mM EDTA, 10 mM dithiothreitol, 1 mM 3-isobutyl-1-methylxanthine, 125 mM KCl, 35 mM b-glycerophosphate, 0.1 mg/ml trypsin inhibitor, 1 μM antipain, 1 μM E64, and 0.4 mM PMSF. The lysate was then sonicated for 10 s for 3 times following centrifugation at 14,000 rpm for 15 ml at 4° C. The supernatant (7.5 μg in 20 μl of lysis buffer) was used for PKG activity measurement. The assay was carried out in a total volume of 50 ml containing 150 mM BPDEtide (Calbiochem), 10 mM HEPES, 35 mM b-glycerophosphate, 4 mM magnesium acetate, 5 mM PKI (a synthetic protein kinase A inhibitor, Calbiochem), 0.5 mM EDTA, 200 mM ATP, and 2 mCi of [γ-32P]ATP (specific activity 3,000 Ci/mmol, GE Healthcare) with or without addition of 2.5 μM cGMP. The mixture was incubated at 30° C. for 12 min and terminated by spotting 40 ml aliquots of mixture on phosphocellulose papers (P81, Whatman). Following 4 washing with 75 mM of ice-cold phosphoric acid solution, the papers were then dried and counted in a liquid scintillation counter. 5 μl of the reaction mixture was directly spotted on the phosphocellulose paper without washing for determination of the γ-32P-ATP specific activity. PKG activity is expressed as picmoles of 32P incorporated into PKG substrate per min per microgram protein.

NO Measurement. eNOS-derived NO from human lung samples was also determined using the three-electrode system (1). Briefly, with the aid of a micromanipulator, the NO sensor was carefully placed on the surface of the lung slice and the baseline was recorded. To determine the lung eNOS activity, the samples were subsequently incubated in a bath containing HBSS, and iNOS and nNOS inhibitors for 20 min. After addition of 1 mM L-Arginine, NO release was recorded and the maximal current at 20 s was calculated for eNOS-derived NO production. Similarly, iNOS-derived NO was also determined with the three-electrode system. Following 20 min incubation of eNOS and nNOS inhibitor (L-NNA, 4 μM), L-Arginine was added and NO release was recorded. The maximal current at 20 min was calculated for iNOS-derived NO production.

Detection of Protein S-nitrosylation. S-nitrosylation was detected with the S-Nitrosylated Protein Detection Assay kit (Cayman Chemical) following manufacture's instruction. Briefly, freshly isolated mouse lung tissue under weak fluorescent light condition was homogenized in Buffer A containing blocking reagent and precipitated with ice-cold acetone. The precipitates were then resuspended in Buffer B containing reducing and labeling reagents and incubated for 1 h at room temperature. All these procedures were performed under weak fluorescent light condition. And then, the biotin-labeled proteins were precipitated with ice-cold acetone and resuspended in washing buffer. 10 μg of each sample was used for direct Western blotting analysis of total S-nitrosylation. The same blot was blotted with anti-GAPDH for loading control. To detect PKG-1 S-nitrosylation, the samples (300 μg each) were immunoprecipitated with anti-PKG-1 (2 μg) overnight and then detected for S-nitrosylation by Western blotting analysis. The same blot was blotted with anti-PKG-1 for detection of PKG-1 expression.

In Vivo Gene Delivery to Lungs. WT and Cav1^{-/-} mice were randomized into two groups for AdvPKG and AdvLacZ, respectively, and anesthetized for delivery of recombinant adenovirus. While breathing spontaneously, each mouse was nebulized with 75 p. 1 of sterile PBS solution containing 1.5×108 pfu of recombinant adenovirus expressing either human PKG-1 (a generous gift from Dr. K. D. Bloch at the Cardiovascular Research Center and Department of Anesthesia, Massachusetts General Hospital) or LacZ by use of an intratracheal microspray device through the mouth (MicroSprayer, Penn-Century Inc.). 7d after nebulization, mice were anesthetized for measurements of RVSP and PVR. Lungs were collected for Western blot analysis.

Histology and Imaging. Lung tissues were fixed and processed for H & E staining and immunofluorescent staining. for 5 min by instillation of 10% PBS-buffered formalin through trachea catheterization at a transpulmonary pressure of 15 cm H2O, and then overnight at 4° C. with agitation. After paraffin processing, the tissues were cut into semi-thin 4 to 5 mm thick, and stained with H & E for histological analysis. For immunofluorescent staining, antigen retrieval was performed by incubating the slides in 10 mM sodium citrate (pH 6.0) at 95° C. for 10 min. After 1 h incubation at room temperature in a blocking solution containing 2% bovine albumin serum, 0.1% Triton X-100, and 2% normal goat serum, the sections were incubated for 2 h at room temperature with anti-smooth muscle a-actin mAb (1:400, Sigma-Aldrich) and then 1 h with FITC-conjugated goat anti-mouse IgG (1:250, Sigma-Aldrich). Nuclei were counterstained with DAPI. The anti-a-SMA-positive pulmonary arterial vessels per field (200×) were counted based on the diameter (<40 μm versus>40 μm). Twenty fields per section were randomly identified and counted. To examine tyrosine nitration in the pulmonary vasculature, cryosections of mouse lung tissues were fixed with 4% paraformaldehyde and then immunostained with anti-nitrotyrosine (mouse monoclonal antibody, 1:80, Cayman Chemical) to detect nitration (green) and anti-smooth muscle a-actin (rabbit polyclonal antibody, 1:250, Abcam) to detect muscularized vessels (red). The Nuclei were counterstained with DAPI.

### Example 13

### Methods for Examples 14-17

Animals. To generate the DKO mice, eNOS^{-/-} mice were bred into the background of Cav1^{-/-} mice (The Jackson Laboratory). All mice were bred and maintained in the University of Illinois according to NIH guidelines. Approval for animal care and use for these experiments was granted by the Institutional Animal Care and Use Committee of the University of Illinois at Chicago.

Human Subjects. Human lung tissues were obtained from patients undergoing lung transplantation for IPAH (n=4; age 31.5±19.2 yr; gender, 2M+2F) and from unused donor lungs (n=4; age 36.2±16.1 yr; gender, M). Informed consent and local ethical approval from the Hammersmith Hospitals (Ref. No. 2001/6003) and Royal Brompton & Harefield Hospitals (Ref. No. 01-210) ethics committees were obtained prior to tissue collection.

Molecular Analysis. Western blot analyses were performed using anti-caveolin-1 (1:1000) and anti-iNOS (1:500, Santa Cruz Biotechnology), anti-eNOS (1:1500) and anti-hsp90 (1:1000, BD Biosciences), anti-nitrotyrosine (1:1000, Millipore), anti-PKG-1 (1 mg/ml, a generous gift from Dr. X.-P. Du at Department of Pharmacology, University of Illinois at Chicago), anti-p42/44 and anti-phosphorylated p42/44 (1:1000, Cell Signaling Technology), anti-VASP (1:1000, Axxora, LLC) and anti-VASP phosphoSer239 (1:200, Axxora, LLC). Anti-a-actin (1:4000, Sigma) or anti-GAPDH (1:2000, Santa Cruz Biotechnology) was used as a loading control. To detect PKG-1 tyrosine nitration, protein lysates from either mouse or human lungs (500 μg each) or primary cultures of human pulmonary artery smooth muscle cells (80 μg each) were immunoprecipitated overnight with anti-PKG-1 (2 μg each) and then probed with anti-nitrotyrosine (1:2500, Cayman Chemical).

RNA was isolated using an RNeasy Mini kit including DNase I digestion (Qiagen) and quantitative RT-PCR analysis was performed in ABI Prism 7000 Sequence Detection System (Applied Biosystems) with QuantiTect SYBR Green RT-PCR kit (Qiagen). The sequences of the primers were provided in Supplemental Methods.

NO Measurements. eNOS-derived NO from lung samples was determined with Griess reagent (Promega Co). Samples were incubated in 1 ml F-12 DMEM with inhibitors for iNOS (1400 W, 4 μM) and nNOS (N\acute{ω}-propyl-L-Arginine, 1 μM) for 20 min. 1 mM L-arginine was then added and incubated for 3 h. Aliquots of medium were collected and NO release was determined by measuring the concentration of nitrite and nitrate (NOx) in the aliquot in two steps using the Nitralyzer-II kit (World Precision Instruments, Inc) and Griess reagent. Results were expressed as nM/g lung/h incubation. Total NOx production from mouse lung samples was determined with similar method without incubation of NOS inhibitors. eNOS-derived NO from human lung samples was also determined using the three-electrode system as detailed in Supplemental Methods.

Hemodynamic Measurements. RVSP was determined with a 1.4 F pressure transducer catheter (Millar Instruments) and the Acknowledge software (Biopac Systems, Inc.). Briefly, the 1.4 F pressure transducer was inserted through the right external jugular vein of anesthetized mice (100 mg ketamine/5 mg xylazine/kg BW, i.p.) and threaded into the right ventricle. RVSP was then recorded and analyzed with the Acknowledge Software.

PVR was measured. Briefly, the isolated lungs was ventilated (120/min and end expiratory pressure of 2.0 cm H2O) and perfused at constant flow (2 ml/min), and venous pressure (+4 cm H2O) with RPMI 1640 medium supplemented with 3 g/100 ml of BSA. Pulmonary arterial and venous pressures were monitored using pressure transducers (Model P23XL-1; Grass Instrument Co.). PVR was calculated from (Ppa−Ppv)/(Q/100 g), where Ppa and Ppv are pulmonary arterial and venous pressures, and Q is flow (2 ml/min).

PKG Kinase Assay. In vitro activity of PKG was determined by measuring the transfer of the [g-³²P] phosphate group of ATP to the specific PKG substrate, BPDEtide (Calbiochem) in the absence or presence of exogenous 2.5 mM cGMP as described in Example 13. PKG activity is expressed as picmoles of ³²P incorporated into PKG substrate per min per microgram protein.

Identification of Nitrated Tyrosine Residues of PKG-1a. Tyrosine residue was mutated to Phenylalanine by site-directed mutagenesis following manufacture's instruction (Stratagene). Myc-tagged wild-type and mutant PKG-1a were overexpressed in human lung microvascular endothelial cells and cell lysates were then immunoprecipitated with anti-Myc for 4 hr at RT. The same amount of immunoprecipitates was incubated with either 100 μM of peroxynitrite (in 0.1N NaOH) or the same amount of 0.1N NaOH (control). Following 14 min incubation in 50 mM K₂HPO₄ buffer at RT, kinase activity was then measured as described above.

Histology and Imaging. Lung tissues were fixed and processed for H & E staining and immunofluorescent staining.

Statistical Analysis. Data are presented as mean±SD. Statistical significance of differences between group means was determined using an unpaired two-tailed Student's t-test; P<0.05 was considered significant.

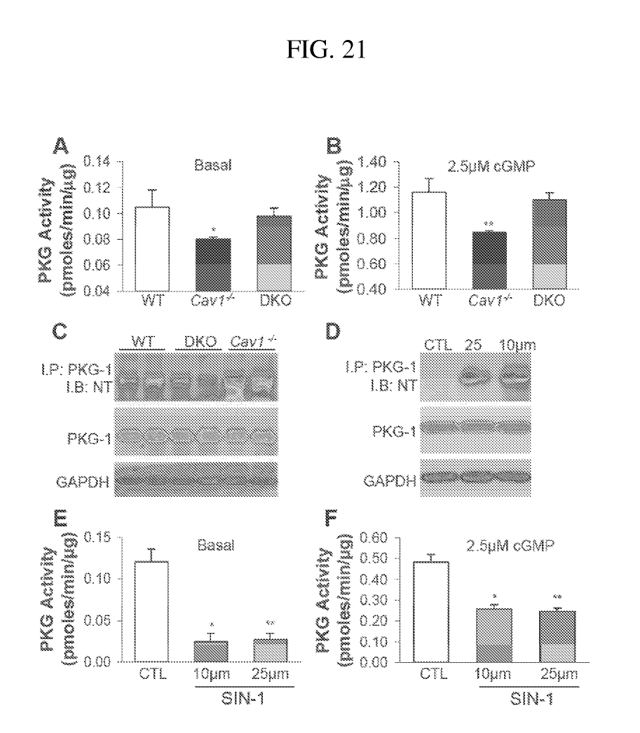

FIG. 21A-B shows in vitro PKG activity in lung lysates during basal state (A) and following addition of 2.5 mM cGMP (B). Data are expressed as mean±SD (n=3-5). *, P<0.05 Cav1^{-/-} versus WT, or DKO; **, P<0.05 Cav1^{-/-} versus WT, or DKO. (C) Prominent tyrosine nitration of PKG detected in lung lysates from Cav1^{-/-} mice. Lung lysates (150 mg) from WT, DKO, and Cav1^{-/-} mice, respectively, were immunoprecipitated with 4 mg of anti-PKG-1 antibody overnight and PKG tyrosine nitration was detected with anti-nitrotyrosine (NT) by Western blotting. Protein expression of PKG-1 in lung lysates was also determined directly by Western blotting with anti-PKG-1 antibody. The same blot was re-probed with an antibody against mouse GAPDH as a loading control. (D) PKG-1 tyrosine nitration in cultured human pulmonary artery smooth muscle cells following SIN-1 treatment. Subconfluent primary cultures were treated with SIN-1 at the indicated concentrations for 30 min, and each cell lysate (50 mg) was then used for immunoprecipitation and immunoblotting for detection of PKG-1 nitration. Each lysate (15 mg) was also used for direct immunoblotting with anti-PKG-1 and anti-GAPDH, respectively. CTL, control. (E-F) SIN-1 treatment resulted in significant decrease of PKG activity in the basal state (E) and following addition of 2.5 mM of cGMP (F). Data are shown as mean±SD (n=3). *, P<0.001 versus CTL; **, P<0.001 versus CTL.

### Example 14

### Rescue of Pulmonary Vascular Pathology in DKO mice

Studies have demonstrated severe lung hypercellularity and thickening of alveolar septa in Cav1^{-/-} mice; thus, we addressed the possibility that chronically active eNOS in Cav1^{-/-} mice was responsible for the lung pathology in these mice. DKO lungs exhibited normal alveolar-capillary structure and vessel wall thickness in contrast to Cav1^{-/-} lungs. Histological scoring showed normal pulmonary morphology in DKO lungs. To quantify the number of muscularized distal pulmonary arteries, an underlying feature of pulmonary vascular remodeling in PH, lung sections were stained with anti-a-SMA. Cav1^{-/-} lungs exhibited 3-fold increase in muscularized distal arteries (<40 μm in diameter) compared to WT, whereas similar number of muscularized large vessels was seen in both Cav1^{-/-} and WT lungs. DKO lungs exhibited similar number of muscularized distal and large vessels as the WT and eNOS^{-/-} lungs.

To address the molecular basis of the hyperplasia and pulmonary vascular remodeling seen in Cav1^{-/-} lungs, we examined ERK signaling and expression of genes regulating cell cycle progression. FIG. 20 shows western blot analysis of ERK signaling. Phosphorylation of p42/44 mitogen-activated protein kinase was detected with anti-phospho p42/44 in lung lysates (30 μg per lane). The same blot was immunobloted with anti-p42/44 for detection of total p42/44 and with anti-b-actin for loading control. FIG. 20B-D, shows quantitative analyses of gene expression in mouse lungs. Total RNA was isolated from lungs collected from 2 month old mice and mRNA levels of p21Cip1 (B), IGF-I (C) and VEGF-A (D) were analyzed with quantitative SYBR Green RT-PCR assay. mRNA levels of cyclophilin were used for normalization. Data are expressed as mean±SD (n=3-5). *, P<0.01 versus either WT or DKO. Expression of p21Cip1 and growth factors IGF-I and VEGF-A was normalized in DKO lungs.

As shown in FIG. 20A, ERK signaling was activated in Cav1^{-/-} lungs compared to either WT or DKO. Quantitative RT-PCR analysis showed that the mRNA level of p21Cip1, a cyclin-dependent kinase inhibitor, was decreased 5-fold in Cav1^{-/-} lungs compared to WT, whereas p21Cip1 expression in DKO lungs was restored (FIG. 20B). Interestingly, expression of several growth factors such as insulin-like growth factor 1 (IGF-I) and vascular endothelial growth factor-A (VEGF-A) was increased in Cav1^{-/-} lungs, whereas they were normal in DKO lungs (FIGS. 20C and 20D). Thus, activation of ERK signaling and downregulation of p21Cip1 in Cav1^{-/-} lungs may be the result of increased expression of these growth factors in Cav1^{-/-} lungs.

### Example 15

### Tyrosine Nitration of PKG Secondary to Caveolin-1 Deficiency Impairs PKG Activity

NO reacts with superoxide to form the damaging reactive nitrogen species peroxynitrite that modifies proteins and may interfere with their function through tyrosine nitration. Immunostaining of nitrotyrosine, a surrogate measure of peroxynitrite, showed that Cav1^{-/-} lungs had marked nitrotyrosine compared to WT and DKO lungs, indicating the formation of peroxynitrite in Cav1^{-/-} lungs. Prominent nitrotyrosine immunostaining was also evident in Cav1^{-/-} pulmonary vasculature including muscularized distal arteries. Western blotting also demonstrated increased tyrosine nitration of proteins in Cav1^{-/-} lungs whereas no difference in S-nitrosylation of proteins was seen in Cav1^{-/-} and WT lungs.

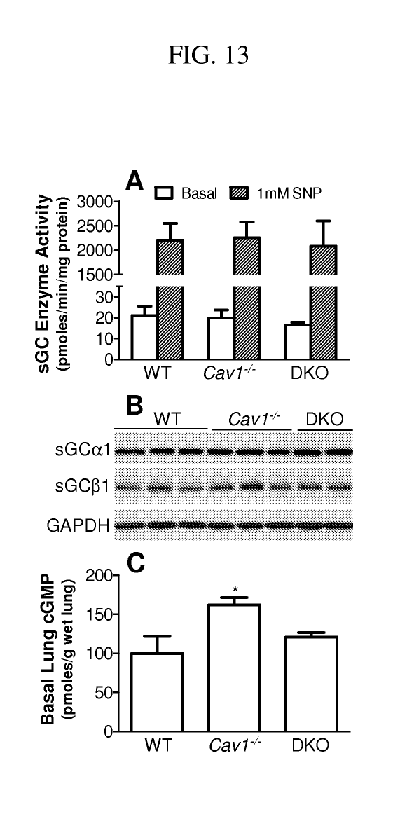

We determined the activities of soluble guanylyl cyclase (sGC) and protein kinase G (PKG), the two major downstream targets of NO signaling, to investigate whether their functions were impaired by tyrosine nitration in Cav1^{-/-} lungs. With respect to FIG. 13, each lung lysate (50 μg), partially purified by 1 h centrifugation at 100,000 g, was used for sGC activity assay at either basal state or after addition of sodium nitroprusside (SNP) at 1 mM, a high dose for maximal activation. Data are expressed as mean±SD (n=3-5). There is no difference in sGC activities at either basal or under maximal stimulation among WT, Cav1^{-/-} and DKO lungs. FIG. 13B shows expression of sGC subunits in mouse lungs. Each lung lysate (20 μg) was used for Western blot analysis of the α1 subunit (sGCα1) and the same blot was blotted with anti-sGCβ1 and GAPDH. We observed no differences in protein levels of either subunit in mouse lungs. FIG. 13C shows increased cGMP production in Cav1^{-/-} lungs. Lungs were lysed with 6% (w/v) trichloroacetic acid and then cGMP concentrations were determined with a cGMP Enzyme Immunoassay Kit (GE Healthcare) following manufacturer's instruction. Data are expressed as mean±SD (n=3-4 per group). *, P<0.05 versus either WT or DKO.

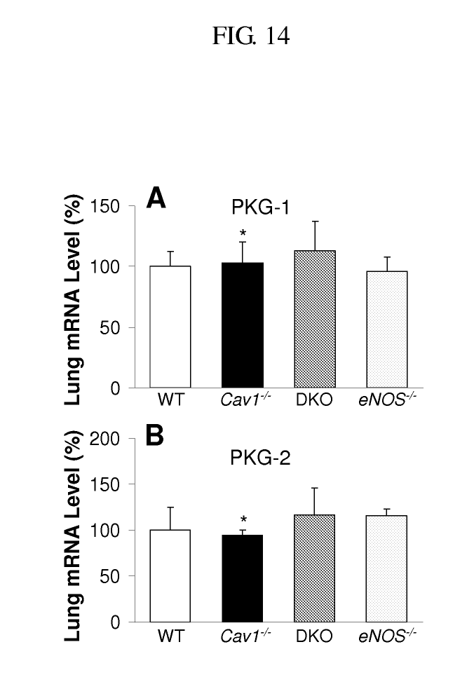

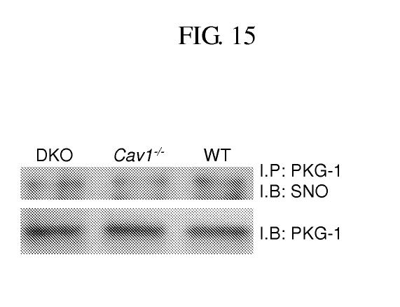

FIG. 13 shows that baseline sGC activity and maximal activity after stimulation with sodium nitroprusside, a NO donor were similar in WT, Cav1^{-/-}, and DKO lungs. In sharp contrast, basal PKG activity in Cav1^{-/-} lungs was 30% less than in either WT or DKO groups (FIG. 21A), and also following the addition of 2.5 μM cGMP (FIG. 21B). PKG dysfunction in Cav1^{-/-} lungs could not be ascribed to reduced cGMP production since Cav1^{-/-} lungs in fact exhibited higher cGMP concentrations (FIG. 13). The protein levels of PKG-1 were also similar in WT, Cav1^{-/-} and DKO lungs (FIG. 21C). Quantitative RT-PCR demonstrated similar mRNA expression of both PKG-1 and PKG-2 in mouse lungs (FIG. 14). These data suggest that impaired PKG activity in Cav1^{-/-} lungs is the result of post-translation modification. Indeed we observed marked nitrotyrosine modification of PKG-1 in Cav1^{-/-} lungs as compared to DKO and WT lungs (FIG. 21C). In contrast, S-nitrosylation of PKG-1 in Cav1^{-/-} lungs was minimal and similar to WT and DKO lungs (FIG. 15).

For FIG. 14, total RNA isolated from lungs was collected from 2 mo old mice and mRNA levels of PKG-1 (A) and PKG-2 (B) were analyzed with quantitative RT-PCR assay. mRNA levels of cyclophilin were used for normalization. Data are expressed as mean±SD (n=3-5). *, P>0.5 Cav1^{-/-} versus either WT, or DKO, or eNOS^{-/-}. mRNA levels of PKG-1 was approximately 2 times of PKG-2 in mouse lungs.

For FIG. 15, biotin-labeled lysates (300 μg per sample) of freshly isolated mouse lung tissue was immunoprecipitated with anti-PKG-1 overnight, and then S-nitrosylation (SNO) was detected by Western blot analysis with avidin-coupled reagents. The same blot was also immunoblotted with anti-PKG-1. Low and similar levels of S-nitrosylation of PKG-1 were detected in lung tissues from WT, Cav1^{-/-}, and DKO.

FIG. 21A-B shows in vitro PKG activity in lung lysates during basal state (A) and following addition of 2.5 mM cGMP (B). Data are expressed as mean±SD (n=3-5). *, P<0.05 Cav1^{-/-} versus WT, or DKO; **, P<0.05 Cav1^{-/-} versus WT, or DKO. (C) Prominent tyrosine nitration of PKG detected in lung lysates from Cav1^{-/-} mice. Lung lysates (150 mg) from WT, DKO, and Cav1^{-/-} mice, respectively, were immunoprecipitated with 4 mg of anti-PKG-1 antibody overnight and PKG tyrosine nitration was detected with anti-nitrotyrosine (NT) by Western blotting. Protein expression of PKG-1 in lung lysates was also determined directly by Western blotting with anti-PKG-1 antibody. The same blot was re-probed with an antibody against mouse GAPDH as a loading control. (D) PKG-1 tyrosine nitration in cultured human pulmonary artery smooth muscle cells following SIN-1 treatment. Subconfluent primary cultures were treated with SIN-1 at the indicated concentrations for 30 min, and each cell lysate (50 mg) was then used for immunoprecipitation and immunoblotting for detection of PKG-1 nitration. Each lysate (15 mg) was also used for direct immunoblotting with anti-PKG-1 and anti-GAPDH, respectively. CTL, control. (E-F) SIN-1 treatment resulted in significant decrease of PKG activity in the basal state (E) and following addition of 2.5 mM of cGMP (F). Data are shown as mean±SD (n=3). *, P<0.001 versus CTL; **, P<0.001 versus CTL.

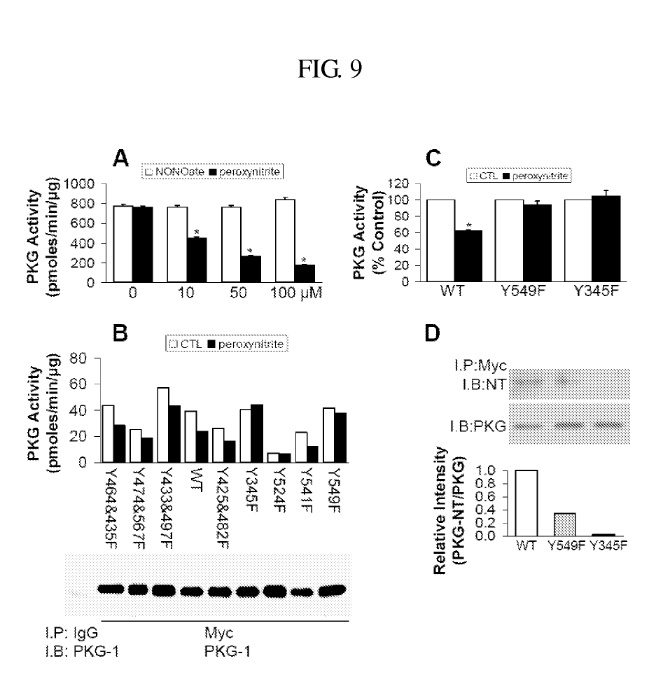

To address the role of peroxynitrite in the mechanism of the observed impairment of PKG activity, we treated human pulmonary artery smooth muscle cells with 3-morpholinosydnonimine (SIN-1), the superoxide and NO donor that forms peroxynitrite simultaneously (30). SIN-1 concentrations as low as 10 mM were shown to cause marked PKG tyrosine nitration (FIG. 21D) and decreased PKG activity (FIGS. 21E and 21F). Furthermore, in vitro treatment of purified PKG-1 with peroxynitrite resulted in decreased PKG activity in a concentration-dependent manner whereas DETA NONOate, a NO donor, even at 100 μM failed to suppress PKG activity (FIG. 9A). FIG. 9A relates to dose-dependent impairment of PKG activity by peroxynitrite. Purified PDG-1 (Promega) was incubated with peroxynitrite at indicated concentrations for 14 min at RT or with DETA NONOate (NONOate) for 30 min in dark at RT. Kinase activity was then assayed. Data is expressed mean+/−SD (n=3). *, P<0.05 versus control (0 μM). FIG. 9B relates to screening of PKG-1a mutants with in vitro kinase assay. At 48 hr post-transfection, myc-tagged WT and PKG-1a mutants were immunoprecipitated with anti-myc beads and aliquoted for incubation with either peroxynitrite (100 μM) or the same amount of 0.1 N NaOH only (CTL). In vitro kinase assay was then performed to determine PKG activity. PKG activity was expressed as pmoles/min/μg cell lysates. Western Blotting was used to detect the protein levels of PKG-1a. FIG. 9C relates to validation of target tyrosine residues. At 48 hr post-transfection, myc-tagged WT and PKG-1a mutants were immunoprecipitated for tyrosine nitration and in vitro kinase assay. Kinase activity following peroxynitrite incubation was normalized to its control (CTL), respectively. Data are expressed as mean±SD (n=3), *, P<0.05 versus either PKG-1a mutant. FIG. 9D relates to diminished tyrosine nitration of PKG-1 mutants. Following 14 min incubation with peroxynitrite (250 μM) at RT, the anti-myc immunoprecipitates were used for Western blotting to detect tyrosine nitration. The intensity of each band of PKG-1 tyrosine nitration was normalized with the intensity of each PKG-1 band, respectively.

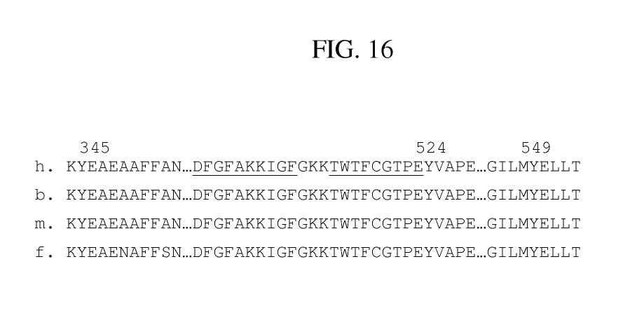

To identify the target tyrosine residues responsible for impairment of PKG activity upon nitration, we mutated all tyrosine residues in the catalytic domain of human PKG-1a into phenylalanine and expressed these myc-tagged PKG-1 mutants with either single or double mutations in human lung microvascular endothelial cells. As shown in FIGS. 9B and 9C, mutation of either tyrosine residue 345 or 549 resulted in no impairment of PKG activity following peroxynitrite treatment in contrast to other mutations and wildtype PKG-1. Accordingly, these PKG-1 mutants exhibited diminished tyrosine nitration in contrast to wildtype PKG-1 following peroxynitrite treatment (FIG. 9D). Both Tyr 345 and 549 are conserved from zebra fish to human PKG-1 (FIG. 16). FIG. 16 shows sequence alignment analysis demonstrating conserved tyrosine residues. Underlined regions indicating the activation loop. h, human, b, bovine, m, mouse, f, zebra fish. These data demonstrate that peroxynitrite formation impairs PKG activity through nitration at tyrosine residues 345 and 549. Decreased kinase activity of PKG-1a mutant with mutation at tyrosine residue 524 was likely ascribed to its location next to the activation loop (FIG. 9B and FIG. 16).

### Example 16

### Nitrative Stress-Mediated PKG Dysfunction Induces PH in Cav1^{-/-} Mice

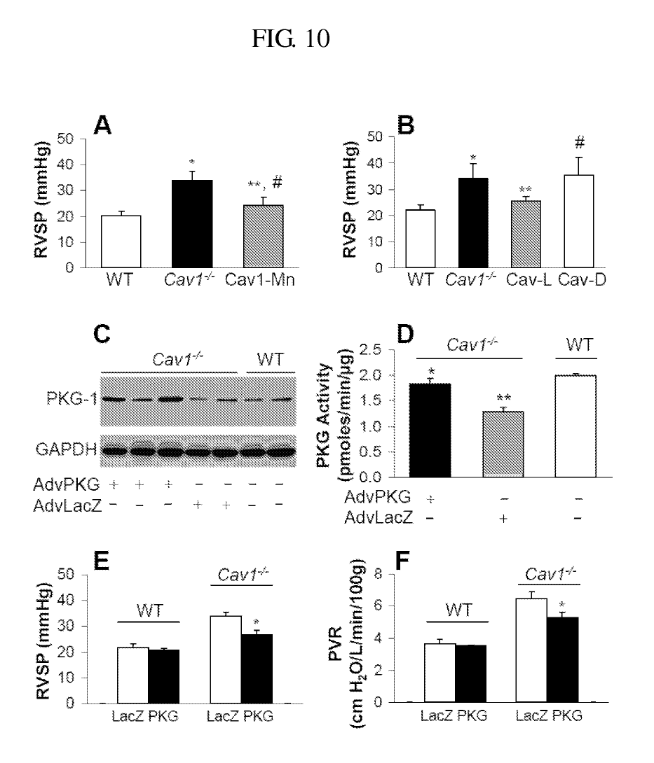

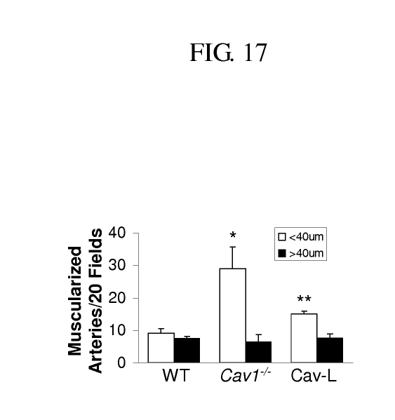

To determine whether tyrosine nitration-mediated impairment of PKG activity was responsible for the development of PH in Cav1^{-/-} mice, we carried out a series of experiments that included blocking peroxynitrite formation and overexpressing PKG. We first determined whether scavenging superoxide by manganese (III) tetrakis (1-methyl-4-pyridyl) porphyrin pentachloride (MnTMPyP, a superoxide dismutase mimetic) could reverse PH in Cav1^{-/-} mice. In this experiment, 8 month old Cav1^{-/-} mice received either saline or MnTMPyP (5 mg/kg, i.p. daily) for 6 wk. We observed that MnTMPyP treatment reduced RVSP (FIG. 10A). We also treated 8 month old Cav1^{-/-} mice with either N-Nitro-L-Arginine Methyl Ester (L-NAME, a NOS inhibitor) or its inactive analog D-NAME in drinking water (1 mg/ml) for 5 wk. Inhibition of NO production by L-NAME treatment also reduced RVSP and the number of muscularized distal pulmonary arteries whereas D-NAME had no effect (FIG. 10B and FIG. 17). These data suggest high levels of NO per se in Cav1^{-/-} mice do not cause PH but superoxide production is also important for its development; i.e., formation of peroxynitrite and resultant protein nitration are required for the development of PH.

Materials and methods for FIG. 10. In FIG. 10A, Cav1^{-/-} mice were treated with either MnTMPyP (5 mg/kg, i.p., daily) (Cav1-Mn) or saline (Cav1^{-/-}) for 6 wk. Data are expressed as mean±SD (n=5 per group). *, P<0.01 Cav1^{-/-} versus WT; **, P<0.05 Cav1-Mn versus Cav1^{-/-}; #, P>0.05 Cav1-Mn versus WT. FIG. 10B relates to inhibition of NOS with L-NAME reversed PH in Cav1^{-/-} mice. Cav1^{-/-} mice received water ad libitum (Cav1^{-/-}, control) or water with 1 mg/ml of L-NAME (Cav-L) or its inactive analog D-NAME (Cav-D) for 5 wk. Data are expressed as mean±SD (n=5-7). *, P<0.01 WT versus Cav1^{-/-};**, P<0.05 Cav-L versus Cav1^{-/-}; #, P>0.5 Cav-D versus Cav1^{-/-}. FIG. 10C-F relates to the restoration of PKG-1 activity in Cav1^{-/-} lungs reversed PH. At 7 d post-administration of recombinant adenovirus expressing either human PKG-1 (AdvPKG) or LacZ, lungs were collected for Western blotting (C), and PKG kinase activity assay (D) following measurements of RVSP (E) and PVR (F). PKG activity is expressed as mean±SD (n=3-4). *, P>0.5 versus WT; **, P<0.05 versus either WT or Cav1^{-/-} treated with AdvPKG (D). RVSP is expressed as mean±SD (n=4-5). *, P<0.05 Cav1^{-/-}-AdvPKG (PKG) versus Cav1^{-/-}-AdvLacZ (LacZ) (E). PVR are expressed as mean±SD (n=3-4). *, P<0.05, Cav1^{-/-}-AdvPKG (PKG) versus Cav1^{-/-}-AdvLacZ (LacZ) (F).

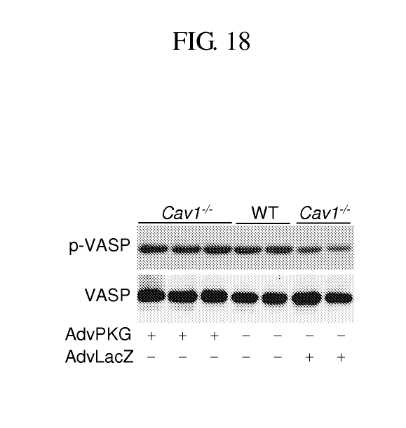

We further tested the hypothesis that nitrative stress-induced PH in Cav1^{-/-} mice was mediated by impaired PKG activity. Recombinant adenoviruses overexpressing human PKG-1 or LacZ were administered into lungs of 10 month old WT and Cav1^{-/-} mice via an intra-tracheal microspray device. At 7 d post-infection, PKG-1 protein expression was elevated by 50% in AdvPKG-treated Cav1^{-/-} lungs compared to AdvLacZ-treated control lungs (FIG. 10C). PKG activities in AdvPKG-treated Cav1^{-/-} lungs were restored to a level similar to WT (FIG. 10D). PKG-mediated phosphorylation of its substrate vasodilator stimulated phosphoprotein (VASP) at residue Ser 239 was also normalized in AdvPKG-treated Cav1^{-/-} lungs (FIG. 18). Accordingly, we observed that restoration of PKG-1 activities in Cav1^{-/-} lungs resulted in a significant decrease in RVSP and PVR whereas minimal changes were seen in WT mice (FIGS. 10E and 10F).

Materials and methods for FIG. 18. 7d post-recombinant adenovirus administration, mouse lung tissues were collected and lysated for Western blot analysis. Anti-VASP phosphoSer239 (p-VASP) was used to detect PKG-mediated phosphorylation of VASP at residue Ser239, and the same blot was immunoblotted with anti-VASP. Decreased VASP phosphorylation was detected in Cav1^{-/-} lungs compared to in WT lungs although similar VASP expression was expressed. AdvPKG administration restored PKG-mediated VASP phosphorylation in Cav1^{-/-} lungs to the levels similar to in WT lungs.

### Example 17

### Increased eNOS Activity and PKG Tyrosine Nitration in Lungs of IPAH Patients

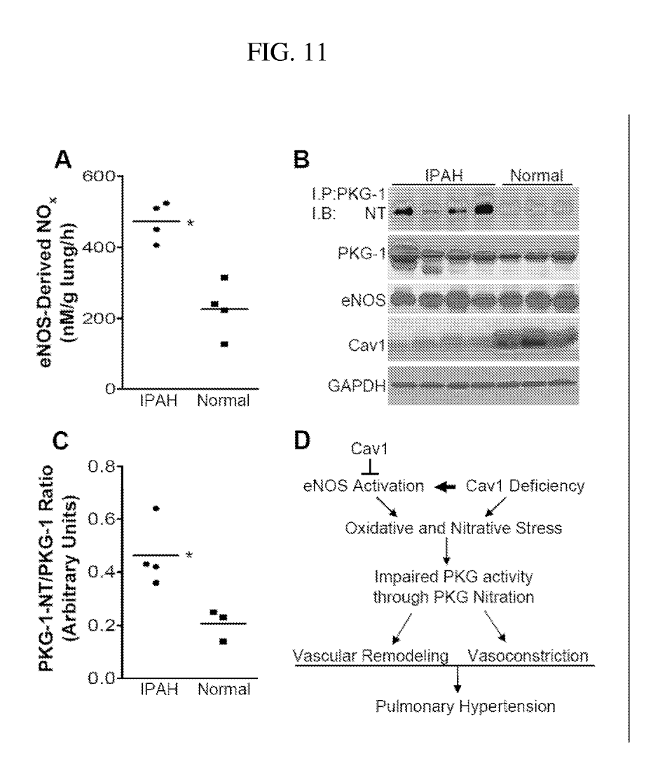

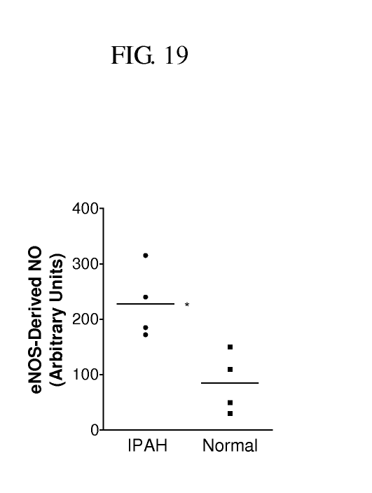

To address the relevance of these observations in mice to the pathogenesis of IPAH in patients, we determined eNOS activity, PKG tyrosine nitration and expression of caveolin-1, eNOS, and PKG-1 in lung tissues from IPAH patients. eNOS activities were increased in IPAH lungs compared to normal lungs (FIG. 11A and FIG. 19), whereas eNOS expression was similar to normal lungs (FIG. 11B). Caveolin-1 expression was decreased in IPAH lungs compared to normal lungs (FIG. 11B) consistent with previous reports (23, 25). Moreover, tyrosine nitration of PKG-1 was markedly increased in IPAH lungs (FIGS. 11B and 11C). We also observed that PKG-1 expression was increased 30% in IPAH lungs (FIG. 11B; IPAH 1.3±0.3 versus normal 1.0±0.07, P=0.12), suggesting a compensatory PKG protein expression in the face of the decreased PKG activity.

Material and methods for FIG. 11. For FIG. 11A, following 20 min incubation with selective inhibitors, lung tissues were incubated with 1 mM L-Arginine for 3 h. eNOS-derived NOx was determined by measuring the concentration of nitrite and nitrate in the medium with the Griess reagent. Bars represent mean. *, P<0.01 versus normal. IPAH lungs produced 2-fold greater eNOS-derived NOx than normal lungs. (B) PKG tyrosine nitration in IPAH lungs. Each lysate (150 μg) was immunoprecipitated with anti-PKG-1 and immunoblotted with anti-nitrotyrosine (NT) for detection of PKG tyrosine nitration. IPAH lungs exhibited prominent PKG-1 tyrosine nitration compared to normal lungs. Protein levels of PKG-1, eNOS, and caveolin-1 were also determined by Western blotting with anti-PKG-1, -eNOS, -caveolin-1 antibody, respectively. Immunoblot of GAPDH was used as loading control. (C) Densitometric analysis of PKG-1 tyrosine nitration. Intensity of each band of PKG-1 tyrosine nitration (PKG-1-NT) was normalized with intensity of each PKG-1 band, respectively. PKG-1 tyrosine nitration increased 2-fold in IPAH lungs compared to normal lungs. Bars represent mean. *, P<0.05 (n=3-4). (D) Proposed model of PKG nitration-mediated PH. Persistent eNOS activation secondary to caveolin-1 deficiency (Cav1^{-/-} mice or IPAH patients) leads to formation of peroxynitrite in the pulmonary vasculature, and impairment of PKG kinase activity through tyrosine nitration. Impaired PKG signaling induces pulmonary vascular remodeling and vasoconstriction, and thereby PH.

While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should be construed broadly and in a manner consistent with the spirit and the scope of the invention herein.

We claim:

1. A method of treating pulmonary hypertension in a subject, comprising administering a PKG-effector agent to a subject in need thereof.

2. The method of claim 1, wherein the PKG-effector agent is selected from the group consisting of a peroxynitrate scavenger, a superoxide scavenger, flavonoid, NOS inhibitor, a PKG activator, a NADPH oxidase inhibitor, a superoxide dismutase activator, a peroxidase activator, a catalase activator, and combinations thereof.

3. The method of claim 3, wherein the peroxynitrate scavenger is selected from the group consisting of uric acid, a plant extracted proanthocyanidin, ascorbate, trolox, glutathione (GSH), Mn (III) tetrakis (4-benzoic acid) porphyrin (MnTBAP), flavonoid, ebselen, catchol (1,2-dihydroxybenzene), kaempferol, galangin, caffeic acid, o-coumaric acid, p-coumaric acid, gallic acid, and ferulic acid.

4. The method of claim 2, wherein the proanthocyanidin is extracted from an arborescent or herbaceous plant species.

5. The method of claim 2, wherein the superoxide scavenger is selected from the group consisting of manganese (III) tetrakis (1-methyl-4-pyridyl) porphyrin pentachloride (MnTMPyP), 1-oxyl-2,2,6,6-tetramethyl-4-hydroxypiperidine (TEMPOL), and NAD(P)H:quinone oxidoreductase 1.

6. The method of claim 5, wherein the superoxide scavenger is MnTMPyP.

7. The method of claim 2, wherein the superoxide dismutase activator is selected from the group consisting of a lipid peroxide, reduced glutathione, and 17β-estradiol.

8. The method of claim 2, wherein the NADPH oxidase inhibitor is selected from the group consisting of apocynin and diphenylene iodonium.

9. The method of claim 2, wherein the peroxidase activator is selected from the group consisting of iron, copper, melatonin, N-acetylcysteine, and 4-hydrobenzoic acid.

10. The method of claim 2, wherein the catalase activator increases catalase expression.

11. The method of claim 10, wherein the catalase activator is an oxidized linoliec acid.

12. The method of claim 11, wherein the oxidized linoleic acid is selected from the group consisting of 13-hydroperoxy-9,11-octadecadienoic acid (13-HPODE), 13-hydroxy-9,11-octadecadienoic acid (13-HODE), hydrogen peroxide, and oxidized LDL.

13. The method of claim 2, wherein the flavonoid is selected from the group consisting of quercetin, rutin, morin, acacetin, hispidulin, hesperidin, and naringin.

14. The method of claim 2, wherein the NOS inhibitor is selected from the group consisting of N omega-nitro-L-arginine, N omega-monomethyl-L-arginine, 1-N^{G} monomethyl arginine (1-NMMA), a caveolin-1 peptide, ARL 17477, and KLYP956.

15. The method of claim 14, wherein the caveolin-1 peptide comprises SEQ ID NO:1.

16. The method of claim 2, wherein the PKG activator is selected from the group consisting of phosphatidylinositol-3,4,5-trisphosphate (PtdIns(3,4,5)P₃), cyclic guanosine 3′, 5′-monophosphate (cGMP), 8-pCPT-cGMP (cGMP derivative), and a cGMP phosphodiesterase inhibitor.

17. The method of claim 16, wherein the cGMP phosphodiesterase inhibitor is selected from the group consisting of sulindac sulfone and OSI-461.

18. The method of claim 1, further comprising administering a PDE5 inhibitor before, after, or at the same time as administering the PKG-effector agent to the subject in need thereof.

19. The method of claim 18, wherein the PDE5 inhibitor is selected from the group consisting of sildenafil, avanafil, tadalafil, acetildenafil, CGMP specific phosphodiesterase type-5, udenafil, vardenafil, and combinations thereof.

20. The method of claim 1, wherein the PKG-effector agent is administered by a route selected from the group consisting of systemic, oral, inhalation, parenteral, nasal, vaginal, rectal, sublingual, and topical.

21. The method of claim 1, wherein the PKG-effector agent is in a formulation selected from the group consisting of a capsule, tablet, a elixir, a suspension, a dry powder, an aerosol, and a syrup.

22. The method of claim 20, wherein the PKG-effector agent is administered by inhalation.

23. The method of claim 1, wherein the pulmonary hypertension is selected from the group consisting of pulmonary arterial hypertension and idiopathic pulmonary arterial hypertension.

24. The method of claim 1, wherein the pulmonary hypertension is secondary to another disease.

25. The method of claim 24, wherein the other disease is selected from the group consisting of pulmonary fibrosis and scleroderma.

26. The method of claim 1 or claim 18, further comprising administering an endothelin receptor antagonist before, after, or at the same time as administering the PKG-effector agent to the subject in need thereof.

27. The method of claim 26, wherein the endothelin receptor antagonist is selected from the group consisting of atrasentan, bosentan, sitaxsentan, and ambrisenten.

28. A method of diagnosing pulmonary hypertension in a subject, comprising providing one or more antibodies that bind to nitrated PKG, contacting the one or more antibodies with a sample from the subject, and identifying the subject as having pulmonary hypertension if the one or more antibodies bind to nitrated PKG and is/are detected in the sample.

29. The method of claim 28, wherein the nitrated PKG is detected using an antibody capable of binding the one or more antibodies that are bound to nitrated PKG.

30. A kit comprising:
(a) a sample collecting means;
(b) means for determining the presence of a PH-marker in the sample; and
(c) a control sample, wherein the control sample does not have a PH-marker.


SEQUENCE LISTING (13 sequences)

SEQ ID NO: 1 - protein, 20 aa, Homo sapiens
DGIWKASFTTFTVTKYWFYR

SEQ ID NO: 2 - DNA, 22 bp, Artificial
  Primer
gacaagaggcccagtacttcct

SEQ ID NO: 3 - DNA, 21 bp, Artificial
  Primer
caatctgcgcttggagtgata

SEQ ID NO: 4 - DNA, 20 bp, Artificial
  Primer
acaggctatggctccagcat

SEQ ID NO: 5 - DNA, 20 bp, Artificial
  Primer
gctccggaagcaacactcat

SEQ ID NO: 6 - DNA, 22 bp, Artificial
  Primer
tccaagatccgcagacgtgtaa

SEQ ID NO: 7 - DNA, 23 bp, Artificial
  Primer
tggcttgtcacatctgcaagtac

SEQ ID NO: 8 - DNA, 22 bp, Artificial
  Primer
accgaatacaggctgacgatgt

SEQ ID NO: 9 - DNA, 22 bp, Artificial
  Primer
gcacggattcattcctttgtgt

SEQ ID NO: 10 - DNA, 22 bp, Artificial
  Primer
ctgtcacagatccaggagattg

SEQ ID NO: 11 - DNA, 22 bp, Artificial
  Primer
atcgccttccttgatgatgcag

SEQ ID NO: 12 - DNA, 22 bp, Artificial
  Primer
ttcagtgtggatttctggtccc

SEQ ID NO: 13 - DNA, 22 bp, Artificial
  Primer
gtcatcatttggtctatcccag